(19) United States
(12) Patent Application Publication
(10) Pub. No.: US 2026/0257017 A1
(43) Pub. Date: Sep. 3, 2026

(54) DOSE FRACTIONING AUTOINJECTOR

(71) Applicant: Congruence Medical Solutions, LLC, Baltimore, MD (US)

(72) Inventors: Gautam Nithyanand Shetty, Hanover, MD (US); Lance Einfeld, Baltimore, MD (US)

(21) Appl. No.: 19/157,512

(22) PCT Filed: Feb. 22, 2024
(86) PCT No.: PCT/US2024/016852

§ 371 Date: Aug. 18, 2025

Related U.S. Application Data
(60) Provisional application No. 63/486,420, filed on Feb. 22, 2023.
(60) Provisional application No. 63/556,511, filed on Feb. 22, 2024.

(51) Int. Cl. A61M 5/20; A61M 5/32

(52) U.S. Cl. CPC A61M 5/2033; A61M 5/3204; A61M 5/3243; A61M 2205/58

(57) ABSTRACT

Autoinjector configured to allow the user to pause the delivery of a drug once the autoinjector is first activated and to resume the drug delivery thereafter. Dose fractioning autoinjector may be operated to deliver the total desired drug dose in multiple stages and/or at multiple injection sites.

This patent disclosure claims priority to U.S. Provisional Applications No. 63/486,420 (filed Feb. 22, 2023) and No. 63/556,511 (filed on Feb. 22, 2024). The contents of the applications listed in this paragraph are expressly incorporated herein by reference in their entirety.

### FIELD OF INVENTION

This disclosure relates generally to injectable drug delivery devices as well as corresponding methods and manufacture. More specifically, this disclosure relates to injectable drug delivery devices configured to allow the user to self-administer a medicament in multiple injection stages and/or sites.

### BACKGROUND

Any discussion of the prior art throughout the specification should not be considered in any way as an admission that such prior art is widely known or forms part of common general knowledge in the field.

Injectable drug delivery devices designed for self-administration can enable patients to administer their own drug treatment injections in a non-clinical setting (for example, at home) or any other setting where a professional healthcare provider may not be able to assist. Conventional syringes require a user to provide the force necessary to administer the injectable drug. This force is characterized using the Hagen Poiseuille equation. To aid the user, self-administration drug delivery devices have included a stored energy source such as compressed springs to provide the force necessary to inject the drug. Less commonly, some self-administration drug delivery devices have also relied on an electromechanical source of power to drive the injection of the drug. This category of self-administration drug delivery devices includes autoinjectors and wearable body injectors (patch pumps).

Since their introduction in the 1970s, autoinjectors have been adapted for use to deliver an increasing number of drugs. The development of pharmaceuticals and biologics as injectables has placed increasing demands on the performance of autoinjectors. Increasingly, drug formulations are being made more viscous due to, for example, higher strength (concentration) biologics, larger injection volumes, and/or long-acting formulations. Higher drug formulation viscosity requires higher injection force or involves longer injection times. In most conventional, spring-based autoinjectors, a stronger spring would need to be employed, which in turn would cause the autoinjector size to increase. Larger autoinjectors are inherently not discreet, which may be undesirable from the user perspective. In addition, larger autoinjectors can cause instability during the injection procedure. Spring-based autoinjectors have been reported to have breakage of drug filled glass syringes. Impact shock to glass syringes from springs upon their release would increase in amplitude with stronger springs. Hence, the path to incorporating stronger springs for viscous formulations may be problematic.

Electromechanical power based autoinjectors are better positioned to provide a more compact device, while providing additional force for viscous formulations. These autoinjectors can be expensive, however. As a result, they are more practical as reusable autoinjectors because of costs and disposal issues.

More recently, compressed gas has been used as a power source in an autoinjector involving miniaturized compressed gas cylinders. These compressed gas autoinjectors, however, present numerous design challenges. The compressed gas cylinder is typically made of a metal with a welded seal. Breaking the welded seal releases the gas, which in turn is directed to advance a plunger rod sliding in a hermetically sealed cylinder; the plunger rod in turn pushes the plunger stopper to inject the drug. Most single-use syringe autoinjectors include this plunger rod.

Since release of the compressed gas involves breaking a welded seal, high actuation forces are required. Hence, compressed gas autoinjectors typically require incorporation of a levered actuator mechanism to make it practical for use. In addition, extensive plumbing is involved to transport the compressed gas to the plunger stopper.

It is also important to ensure that the chamber in which puncturing pin used to break the welded seal operates is hermetically sealed and that the transport of compressed gas to the syringe occurs with no leaks. Leakage of compressed gas during storage and during the injection step are primary complaints of compressed gas powered autoinjectors in the prior art. The compressed gas is typically an inert gas such as nitrogen, carbon dioxide or argon or the like. In some designs, the release of compressed gas can also cause recoil, which may result in accidental removal of the injection needle from the injection site.

When a plunger rod advanced by compressed gas is employed, the applied force on the plunger stopper can be large. If this applied force is not coaxial, the plunger rod could blow by the plunger stopper resulting in an error in drug delivery, breach of container closure integrity and/or damage to the autoinjector during the injection.

Maintenance of flow rate can be achieved by either ensuring that the drug volume is much lower than total volume that the gas from the compressed gas source can occupy after it is punctured. Another approach is to incorporate a dual phase gas in compressed gas chamber.

Despite the foregoing, compressed gas as power source has significant advantages. The power source has a compact size compared to compression springs. Also, as the injection volume increases and syringe cross-section increases, the available force to drive the plunger stopper in the syringe using a compressed gas source increases for the same pressure. An autoinjector with a compressed gas power source is more practical to be disposable, which may be a benefit with several drugs.

The development of autoinjectors also depends on a changing user environment. The trend in treatment settings continuing to move away from hospitals and clinics to patients' homes means that more people will use autoinjectors to treat health conditions themselves. As such, an important design demand is for autoinjectors to ensure they can be used in an error-free manner.

In particular, new users of autoinjectors inherently have difficulty using autoinjectors in an error free manner. One study showed that 69% of study participants prematurely removed the autoinjector from the injection site before the injection was complete when operating with no instructions. This can be particularly problematic for infrequently injected drugs; patients in this case may not have access to a replacement. This lost dose is driven by the fact that needle-safety mechanism is actuated immediately after the autoinjectors is removed from the injection site. The drug is expelled out of the autoinjector even though the needle safety shield is locked. This locking occurs irrespective of whether the complete dose is administered or not. Addressing this technology gap can alleviate a significant therapy compliance burden.

In addition, there is increasing consideration to move injectable drugs from the hospital to home setting. Intravenous administration requires skilled healthcare professional to set up. Subcutaneous or intramuscular injections on the other hand can be either self-administered by administered by a caregiver at home. Therefore, enabling subcutaneous or intramuscular injection for currently drugs currently injected intravenously can facilitate shift of treatment delivery from hospital to home setting. This would be a significant benefit to healthcare systems and to patients. The volume of drugs injected intravenously is higher than that typically injected subcutaneously or intramuscularly. Therefore, shifting away from intravenous injections would require concentrating the drug solution to decrease the injection volume, which in turn could significantly increase the drug solution viscosity, but yet involve injection volumes greater than those typically injected in the subcutaneous or intramuscular space. An enzyme hyaluronidase has been used to facilitate larger volume injections in the subcutaneous space by temporarily breaking down elements of the subcutaneous tissue. This requires coformulation of the drug with the enzyme creating additional complexity and cost.

The ability for the autoinjector to pause an injection could enable fractions of the dose to be injected at multiple subcutaneous (or intramuscular) injection sites without causing discomfort to a patient from a large volume injection. While this injection regime could be with or without co-formulated hyaluronidase, the pause feature in an autoinjector provides an option for large dose delivery without the need for hyaluronidase. An additional benefit with fractioning and delivering the dose across multiple injection sites is improved kinetics of the drug making bioavailability faster because of higher injection area to volume ratio at each injection site. Deliberately deploying the disclosed Injection Pause™ feature to facilitate large volume injections in the subcutaneous or intramuscular space could enable the shift from intravenous to subcutaneous or intramuscular, and hence enable shift of drug delivery from hospital to home (non-hospital) setting.

There are currently two categories of autoinjectors—single-use (or disposable) autoinjectors and reusable autoinjectors. The pause feature could also enable another category of autoinjectors—a multi-use autoinjector. This multi-use autoinjector would necessarily incorporate a prefilled drug cartridge as the primary container. The autoinjector would incorporate a stop dose corresponding to fraction of the total drug filled in the cartridge. This fraction would be equal to the indicated dose required to be delivered. At the dose stop, the injection needle is wither configured to be removed from the drug cartridge or the mechanism providing the injection force (either compressed gas or compression spring) is either dissipated or counterbalanced by an at least an equal opposing force. A multi-use autoinjector has significant sustainability advantages without the cost disadvantage or reusable autoinjectors.

Autoinjectors contain a drug container prefilled with the drug intended to be injected into the patient. The drug container is typically a prefilled syringe or a prefilled cartridge. The process of making an autoinjector available as a prefilled syringe or cartridge involves significant effort given that the drug should demonstrate compatibility with the drug container over its intended shelf-life. Elaborate efforts may be required to integrate this prefilled drug container (syringe or cartridge) with a single use autoinjector effectively.

Given the investments required to undertake the activities described, currently autoinjectors are only used in drug molecules in later stages of development i.e., Phase 3 or at launch or as part of lifecycle management once the drugs are approved.

Autoinjectors have numerous known benefits to users and to the healthcare system enabling administration of drugs away from a hospital or requiring a healthcare professional. However, these benefits are unrealized where an autoinjector option is unavailable due to financial barriers or drug incompatibility with the drug container. In addition, autoinjectors would be helpful in injection of drugs that would be hard-to-inject manually-such as viscous drugs. These drugs would need to undergo reformulation to continue development or stop its development.

An autoinjector that does not require the investments or efforts to establish long-term drug-container compatibility and that simplifies the drug filling and integration operation would provide the opportunity to introduce an autoinjector drug delivery device option earlier in the drug development cycle or for drugs that can never be available in a prefilled format.

Accordingly, an autoinjector capable of delivering a broad range of medicaments effectively, providing the flexibility of staged delivery, reducing user error, and adapting to cost and drug-container compatibility demands, would be advantageous.

### SUMMARY OF THE INVENTION

It is an object of the present invention to overcome or ameliorate one or more of the disadvantages of prior art, or at least to provide a useful alternative.

The present invention includes injectable drug delivery devices configured to allow the user to self-administer a medicament in a staged delivery. This Injection Pause™ mode of delivery facilitates the options of conventional delivery of the total available dose in one stage at one injection site, or the flexible delivery of the total available dose in multiple stages (dose fractioning) and/or multiple injection sites. This capability further allows delivery of the total available dose over multiple injection sites in the same sitting or multiple injections over multiple days. The Injection Pause™ delivery capability has the added benefit of enhancing the probability that a user inexperienced generally with autoinjector operation will administers the complete prescribed dose despite lack of familiarity with operating an autoinjector.

An autoinjector as disclosed here provides a drug cartridge with a needle introduced in it just prior to dose delivery. To ensure the cartridge entry point is sanitized before needle pierces the cartridge septum to access the enclosed drug, a reservoir of sanitizing agent (for example, isopropyl alcohol) is disposed between the needle and the cartridge septum. The reservoir has a removable seal disposed on the cartridge septum side. The reservoir is part of the needle shield subassembly. The reservoir contents (sanitizing agent) contact the cartridge septum upon removal of seal. This seal could be removed when user removes the cap thereby exposing the sanitizing agent and optionally concurrently contacting the cartridge septum. On the axially opposite site is another seal which is pierced by the non-patient end of the injection needle when the needle shield retracts.

The needle is secured by and axially keyed to needle shield assembly, which is removable for the embodiment where injectable drug is administered over multiple days/multiple injection sites and through multiple needles.

The needle shield subassembly is provided sterile or sterilized prior to attachment to the autoinjector. Sterility is breached only once the cap is removed.

The autoinjector features disclosed here may be adapted to both spring (compression or constant force) and compressed gas powered autoinjectors.

### DESCRIPTION OF THE FIGURES

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 shows progressive side elevational schematic views of an autoinjector disclosed here and shows deployment and retraction of a needle shield during the administration of an injectable drug by the autoinjector. Various stages of operation are provided, marked as (1) As Shipped; (2) Cartridge Punctured; (3) Start of Dose; (4) End of Dose; (5) and End of Procedure. Also provided is a view of the Needle Shield with the Injection Needle removed.

FIG. 2 shows the cover and housing of an autoinjector disclosed here and the components within the autoinjector when the cover and housing are removed.

FIG. 3 shows progressive side elevational schematic views of an autoinjector disclosed here and shows the interaction of the components of a needle shield, needle holder pin, cartridge, needle holder, and spring, during the administration of an injectable drug by the autoinjector. Various stages of operation are provided, marked as (1) As Shipped; (2) Cartridge Punctured; (3) Start of Dose; (4) End of Dose; (5) and End of Procedure. Also provided is a view of the Needle Shield with the Injection Needle removed.

FIG. 4 shows progressive side elevational schematic views of an autoinjector disclosed here and shows the interaction of the components of a needle shield, needle disk, and slider, during the administration of an injectable drug by the autoinjector. Various stages of operation are provided, marked as (1) As Shipped; (2) Cartridge Punctured; (3) Start of Dose; (4) End of Dose; (5) and End of Procedure. Also provided is a view of the Needle Shield with the Injection Needle removed.

FIG. 5 shows progressive side elevational schematic views of an autoinjector disclosed here and shows an exemplary mechanism to pause a spring powered injection that includes the interaction of the components of an indicator, ratchet, wire, spring, and plunger rod, during the administration of an injectable drug by the autoinjector. Various stages of operation are provided, marked as (1) As Shipped; (2) Cartridge Punctured; (3) Start of Dose; (4) Partial Dose; (5) Paused Dose; (6) Resumed Dose; (7) End of Dose; (8) and End of Procedure.

FIG. 6 is an exploded, isometric view of an autoinjector disclosed here with components cross-referenced by number designation as described further infra.

FIG. 7 depicts multiple views of the Puller component of an autoinjector disclosed here.

FIG. 8 depicts multiple views of the Safety component of an autoinjector disclosed here.

FIG. 9 depicts multiple views of the Locking Sleeve component of an autoinjector disclosed here.

FIG. 10 depicts multiple views of the Needle Disk component of an autoinjector disclosed here.

FIG. 11 depicts multiple views of the Follower component of an autoinjector disclosed here.

FIG. 12 depicts multiple views of the Cam component of an autoinjector disclosed here.

FIG. 13 depicts multiple views of the Indicator component of an autoinjector disclosed here.

FIG. 14 depicts multiple views of the Plug component of an autoinjector disclosed here.

FIG. 15 depicts multiple views of the Brace component of an autoinjector disclosed here.

FIG. 16 depicts multiple views of the Front Housing component of an autoinjector disclosed here.

FIG. 17 depicts isometric external views of the Syringe Assembly and Subassemblies of an autoinjector disclosed here.

FIG. 18 depicts cross-sectional views of an autoinjector disclosed here to reveal internal components.

FIG. 19 depicts progressive external front views of an autoinjector disclosed here that show the Injection Pause™ feature and stages of use.

FIG. 20 depicts progressive internal side views of an autoinjector disclosed here with select internal components relevant to gas flow and sealing for the Injection Pause™ feature during stages of use.

FIG. 21 depicts progressive internal side views of an autoinjector disclosed here with select internal components relevant to the wire path of the Injection Pause™ feature and stages of use.

FIG. 22 depicts isolated components of an autoinjector disclosed here relevant to the Cam actuation of the Injection Pause™ feature during steps of use.

FIG. 23 depicts progressive internal front views of an autoinjector disclosed here with select internal components relevant to the lockout of the Injection Pause™ feature and stages of use.

FIG. 24 depicts progressive front external views of the Syringe Assembly Steps.

FIG. 25 depicts isolated components of an autoinjector disclosed here which comprise the rotational lock for Syringe Assembly.

FIG. 26 depicts isolated components of an autoinjector disclosed here which comprise the rotational lock for Syringe Assembly.

FIG. 27 depicts isolated components of an autoinjector disclosed here showing the Safety and Needle Disk Connection during Syringe Assembly.

FIG. 28 depicts progressive front views of a drug being loaded into an autoinjector disclosed here.

FIG. 29 depicts progressive front views of a drug being loaded into an autoinjector disclosed here where the connector is configured and bypasses the plunger stopper.

### DETAILED DESCRIPTION OF EXEMPLARY EMBODIMENTS

This disclosure relates generally to injectable drug delivery devices as well as corresponding methods and manufacture. More specifically, this disclosure relates to injectable drug delivery devices configured to allow the user to self-administer a medicament in multiple (one or more) injection stages and/or sites.

An embodiment disclosed here is an autoinjector. In general, an autoinjector injects drug by application of a driving force to the movable component in a drug container such as syringe or a cartridge. The force in most disposable autoinjectors is provided either decompression of a spring (compression or constant force) or decompression of a compress gas. The application of this driving force, and hence the injection, can be triggered by depressing a needle shield or by actuating a button or switch. Once triggered, the driving force is applied, and the drug is injected automatically. To pause the injection at any point before the complete dose is delivered, the device must either counterbalance the driving force with an opposite force, or stop applying the driving force.

In spring-based autoinjectors, once the spring is released from its compressed state, it will continue to apply the driving force as it decompresses, until the injection is complete. Therefore, pausing injection with a spring-based autoinjector requires the application of a counterbalancing force. This counterbalancing force may include, but is not limited to, a physical stop, friction, or hydraulics.

Embodiments of a physical stop may involve a linear pattern of complementary features placed on either a driving component or an anchoring component such that an interlock occurs between the complementary features on each part. When interlocked, the anchoring component would support/counterbalance the driving force of the driving component and hence pause the injection. One such embodiment was disclosed in the original full application. Similar to a ratcheting mechanism, a linear pattern of features is indicated to allow for pausing the injection at any point in its duration. The components could be actuated between the interlocked state and the unlocked state to pause and resume injection respectively. This may be achieved by relative axial translation or rotation between the driving and anchoring components. The driving component may be travelling axially while the anchoring component can either be rotationally, radially or axially introduced into the interlocking position, bringing the driving component to an abrupt stop. The anchoring component could then return to its previous position or another position to resume travel of the driving component.

Another embodiment of a physical stop may involve a driving component or a component coupled to it, is axially stationary and rotating instead of travelling axially. The rotating component would now involve a circular pattern of features or a cavity that interlocks with complementing features on the anchoring component. Locking and unlocking may be again achieved by translation or rotation of the anchoring component relative to the driving/driven component.

Autoinjector embodiments reliant on friction to pause the injection would function similar to embodiments using a physical stop. The anchoring component would be translated or rotated into place to interfere with driving component. But differences arise in how the components interface. While a physical stop relies on blocking a moving components path, a frictional stop may rely on jamming or clamping down on a moving component to prevent motion such that the driving force (or injection force) is always less than the frictional braking force. Embodiments may involve anchoring components constructed from high friction materials pressing against driving/driven components with force, like a brake. The resulting friction force would equal or exceed the driving force and bring the moving component and injection to a stop. The anchoring components could then be removed to remove the frictional braking component and hence to again allow free motion of the driving component to resume injection. Frictional embodiments may also rely on force amplification or mechanical advantage to achieve the braking forces necessary.

An autoinjector embodiment with pause feature realized utilizing hydraulics is also possible. One such embodiment may feature a cylindrical chamber filled with a plunger at one end and a valve at the other. The chamber would also be filled with an incompressible fluid. The plunger in the cylindrical chamber would be driven by the motion of an injection driving component. If the plunger advances while the valve is open, fluid would flow out of the chamber to another area, allowing for injection to occur. With the valve shut, the incompressible fluid would instead be trapped, and motion of the plunger would halt, hence pausing the injection. Actuation of the valve would allow control over starting and stopping of the injection.

An autoinjector embodiment with pause feature may be achieved with a compressed gas based autoinjector. In this regard, pausing embodiments of compressed gas based autoinjectors can also be achieved by removing or dissipating the driving force without the need to counterbalance the applied force. Methods for removing the driving force include, but are not limited to, disconnecting and resealing the compressed gas source, and diverting the compressed gas source. Embodiments that disconnect and reseal the compressed gas source may rely on a valve or a self-healing/self-sealing septum as disclosed here with the Injection Pause™ feature.

To start injection, such an embodiment would involve opening the valve or penetrating a septum that self-seals around the penetrating component with interfacing components to allow flow of compressed gas to provide the driving force. To pause injection, interfacing components with the source would disconnect the compressed gas source. The valve would shut, or the septum would reseal, containing the remaining compressed gas. Simultaneously the now unconnected interfacing components would dissipate the previously pressurized providing the driving force to vent, pausing the injection in the absence of a driving force. To resume the injection, interfacing components are reconnected to the source.

An alternative embodiment to dissipate the compressed gas source to effect an injection pause, may use a three-way valve that can be toggled between connecting the driving components to the atmosphere or to the compressed gas source. Prior to the start of the injection the valve would be set to connect the driving components to the atmosphere. While in this state the compressed gas source is disconnected from providing a driving force. Actuation of the device to begin injection would then switch the valve to connect the driving components to the compressed gas source. Compressed gas would flow to provide the driving force. To pause injection the valve can be toggled back, again blocking the compressed gas source while simultaneously allowing the pressure powering the driving components to vent and hence dissipating the gas providing the driving force.

Actuation of the injection pause for all embodiments described could be configured to occur passively (without an additional, concerted user step), actively (user needs to undertake a concerted action), or semi-actively.

Passive injection pausing would occur anytime the trigger for injection has been released. This autoinjector embodiment is triggered by depression of its needle shield, or by holding down an additional button, this means the injection pauses if either is released. When no external forces from the user are acting on the autoinjector, the injection pauses.

Active injection pausing instead requires an external force from the user acting on the autoinjector to pause injection. In this embodiment the initial trigger for injection and trigger to pause are decoupled. To pause injection after it has been triggered, a separate button or switch must be actively actuated.

A semi-active injection pausing embodiment would similarly decouple the pause and injection trigger. The device would passively pause, but require user actuation of a button or switch to just be pressed to resume injection.

In each of the embodiments, the needle safety mechanism is couple to the mechanism providing the driving force. The needle safety mechanism is only actuated once the mechanism providing the driving force or another mechanism connected/coupled to it indicates the dose is complete (or nearly complete). The needle safety mechanism upon completion of dose delivery can lockout the autoinjector.

Most currently used single use autoinjectors provide only qualitative visual indication (if at all) of state of the use of the device i.e., visual indication that the injection is occurring and, in some cases, visual indication that injection is complete. These visual indicators are also hard to see in some instances because it involves looking at the syringe/cartridge through cutouts in the autoinjector. It would be beneficial for visual indicator to be easier to see and also include quantitative information regarding progress of dose delivery. for e.g., % or volume of injection completed or remaining. Quantitative metric would be particularly helpful with longer injection times. This quantitative visual indicator would be in addition to the already available qualitative indicator of tracking the amount dose delivery by observing the drug container (syringe or cartridge). Design features for audible cues for start of dose delivery and completion of dose delivery.

This disclosure describes an autoinjector embodiment that comprises a syringe including a barrel, a syringe needle fluidly coupled to an interior of the barrel, and a plunger stopper disposed to translate within the barrel, the plunger stopper radially disposed within the barrel, the plunger stopper separating the interior of the barrel into a drug space configured to contain the injectable drug between the plunger stopper and the syringe needle; a slider co-axially disposed with the syringe, the slider being rotatably disposed relative to the syringe and substantially axially fixed relative to the syringe; a member axially adjacent to the plunger stopper, the axial position of the member being synchronous with the axial position of the plunger stopper within the barrel; a needle safety shield, the needle safety shield being slidably disposed relative to the syringe and the slider whereby an axial force exerted on the needle safety shield slides the needle safety shield relative to the syringe from a shielded position to actuate an applied force onto the plunger stopper wherein the syringe needle is not axially exposed to an injection position wherein the syringe needle is axially exposed to penetrate the injection site; wherein the movement of the needle safety shield is at least partially guided by a pin and track arrangement, one of the pin and the track being formed with the needle safety shield and the other of the pin and the track being formed with the slider whereby movement of the pin within the track controls the position of the needle safety shield relative to the slider, the track including a point-of-no-return, the pin returning the needle safety shield to the shielded position when the axial force is removed when the pin is disposed in the track proximal to the point-of-no-return such that the needle safety shield remains axially movable to the injection position wherein the applied force is removed, and the pin moving to and locking the needle safety shield in the shielded position when the axial force is removed when the pin is disposed in the track distal to the point-of-no-return with rotation of the slider; and wherein rotation of the slider is actuated by movement of the member moving synchronous with the plunger stopper, the member rotating the slider positioning the safety shield pin to be guided along the track including the point-of-no-return to axially lock the needle safety shield in the shielded position as the plunger stopper reaches the end of all dose delivery. This embodiment may also optionally comprise a status indicator indicative qualitatively and quantitatively of the progress of delivery of the injectable drug from the syringe, a status indicator that comprises a dose indicator, and/or a dose indicator that is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe. In this embodiment, the applied injection force may be counterbalanced or dissipated when the pin remains guided in which it was originally disposed, or disconnected (which could include counterbalancing or dissipating.

This disclosure describes a method for dose fractioning by an autoinjector comprising the steps of retracting a needle safety shield from an unused state to actuate an autoinjector; an applied force moving synchronously a member with a plunger stopper disposed to translate within a syringe or cartridge; interrupting autoinjector operation by removing applied force on the plunger stopper translation of needle shield pin along original track of slider to its unused state removing the applied force; resuming autoinjector operation by reapplying force on the plunger stopper, wherein the force is actuated by retraction of the needle safety shield; and completing autoinjector operation by removing applied force on the plunger stopper translation upon orienting a slider to guide a radial pin of the needle safety shield to a point of no return locking out the needle safety shield. This embodiment may also optionally comprise a status indicator indicative qualitatively and quantitatively of the progress of delivery of the injectable drug from the syringe, a status indicator that comprises a dose indicator, and/or a dose indicator that is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe.

This disclosure describes another autoinjector embodiment comprising An autoinjector comprising a cartridge including a barrel, a plunger stopper disposed to translate within the barrel, the plunger stopper radially disposed within the barrel, the plunger stopper separating the interior of the barrel into a drug space configured to contain the injectable drug between the plunger stopper and a cartridge crimped stopper; a slider co-axially disposed with the cartridge, the slider being rotatably disposed relative to the cartridge and substantially axially fixed relative to the cartridge; a member axially adjacent to the plunger stopper, the axial position of the member being synchronous with the axial position of the plunger stopper within the barrel; a needle safety shield, the needle safety shield being slidably disposed relative to the cartridge and the slider whereby an axial force exerted on the needle safety shield slides the needle safety shield relative to the cartridge from a shielded position wherein the injection needle axially keyed to the needle safety shield is not axially exposed to an injection position wherein the injection needle is axially exposed to penetrate the injection site; wherein the movement of the needle safety shield is at least partially guided by a pin and track arrangement, one of the pin and the track being formed with the needle safety shield and the other of the pin and the track being formed with the slider whereby movement of the pin within the track controls the position of the needle safety shield relative to the slider, the track including a point-of-no-return, the pin returning the needle safety shield to the shielded position when the axial force is removed when the pin is disposed in the track proximal to the point-of-no-return such that the needle safety shield remains axially movable to the injection position, and the pin moving to and locking the needle safety shield in the shielded position when the axial force is removed when the pin is disposed in the track distal to the point-of-no-return with rotation of the slider; wherein rotation of the slider is actuated by movement of the member moving synchronous with the plunger stopper, the member rotating the slider positioning the safety shield pin to be guided along the track including the point-of-no-return to axially lock the needle safety shield in the shielded position as the plunger stopper reaches the end of all dose delivery; wherein the force applied to the plunger stopper is counterbalanced by a component introduced in the path of the component coupled to the member when the pin travels from the injection position to the shielded position; and wherein the needle safety shield with interlocked injection needle is removable after the safety shield pin is at the point of no return. In this embodiment, a portion of the member may optionally also be radially disposed within and sealed against the barrel. This embodiment may also further comprise a reservoir containing sanitizer that is disposed between the injection needle and the non-drug side of the cartridge crimped stopper and configured so that the injection needle passes through the reservoir prior to piercing the cartridge crimped stopper to be fluidly couple to the interior of the barrel when the needle safety shield moves from the shielded position to the injection position. This embodiment may also optionally comprise a status indicator indicative qualitatively and quantitatively of the progress of delivery of the injectable drug from the syringe, a status indicator that comprises a dose indicator, and/or a dose indicator that is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe.

This disclosure describes a sterile autoinjector embodiment comprising a preinstalled sterile container operably linked by a connector to a fluid port wherein the connector bypasses a plunger stopper radially disposed within and sealed against the container positioned to deliver a preset injection volume; wherein the connector and fluid port are configured to be removed once the container is filled to the preset injection volume; and wherein the plunger stopper is configured to be radially sealed once the connector is unlinked from the container. In this embodiment, the connector may optionally also be hollow tubing, the sterile container may optionally also be a cartridge, the fluid port may optionally also be a female luer, and/or all other components involved in normal autoinjector operation may optionally also be in a ready-to-use state prior to filling the container with an injectable fluid.

The various embodiments of the autoinjector disclosed here may include one or more of the components numerically designated as follows in the specification, claims, and figures: (1) Device; (2) Needle Shield; (2-1) Needle Shield Track; (2-2) Needle Shield Beam; (2-3) Needle Shield Arms; (2-4) Needle Shield Pins; (3) Drug; (4) Cartridge; (5) Needle Holder; (5-1) Needle Holder Needle; (5-2) Needle Holder Pins; (6) Injection Needle; (7) Housing; (8) Cover; (9) Canister; (9-1) Canister Septum; (10) Window; (11) Plunger Stopper; (12) Needle Shield Spring; (13) Slider; (13-1) Slider Track; (13-2) Slider Keyway; (13-3) Slider Beam; (14) Needle Disk; (14-1) Needle Disk Needle; (14-2) Needle Disk Key; (15) Needle Disk Spring; (16) Device; (17) Indicator; (17-1) Indicator Beam; (18) Wire; (19) Driving Spring; (20) Plunger Rod; (21) Ratchet; (21-1) Ratchet Ramp; (21-2) Ratchet Teeth; (22) Needle Shield; (22-1) Needle Shield Arms; (23) Device; (24) Syringe; (24-1) Syringe Shoulder; (25) Needle Cover; (26) Rigid Needle Shield; (27) Cap; (28) Puller; (28-1) Barbs; (29) Safety; (29-1) Safety Arms; (29-2) Safety Stops; (29-3) Safety Hooks; (30) Spring; (31) Locking Sleeve; (31-1) Locking Beam; (31-2) L-Beam; (32) Needle Disk; (32-1) Air Needle; (32-2) Needle Disk Flanges; (33) Syringe Seal; (34) Follower; (35) Cam; (35-1) Cam Stops; (35-2) Cam Ramp; (36) Plug; (37) Back Cover; (37-1) Track; (38) Indicator Cover; (38-1) Indicator Cover Markings; (39) End Cap; (40) Back Housing; (41) Brace; (41-1) Brace Lip; (41-2) L-Slot; (42) Front Housing; (42-1) Front Housing Lip; (42-2) Housing Window; (43) Window Sleeve; (44) X-ring; (45) O-ring; (46) Seal Insert; (47) Drug Vial; (48) Filling Syringe; (49) Transfer Needle; and (50) Filling Cannula. The respective numerical designations are intended as illustrative guides only to assist with general cross-reference of components and not intended to be definitional. Indeed, components and their numerical designations may represent structural and/or functional elements that may be indistinct or overlap, for example, represent a collective of components or a component of another component.

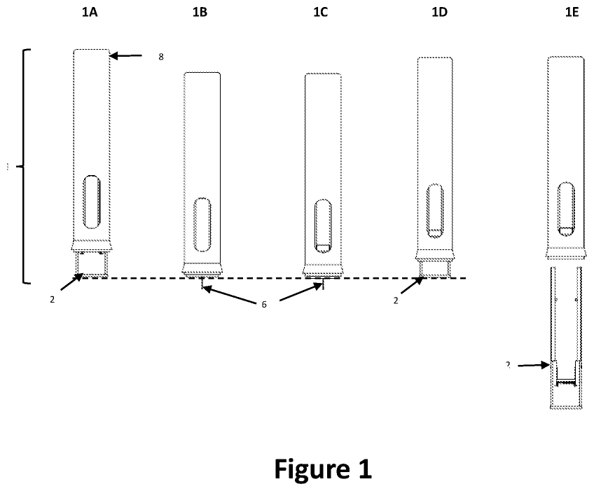

An autoinjector is depicted in FIG. 1 where the Device (1) is received and uncapped to reveal the Needle Shield (2) or Needle Safety Shield in the as shipped state (FIG. 1A) without a cap (not shown). The user then fully, axially depresses the Needle Shield (2) in one continuous motion (FIG. 1B). As the Needle Shield (2) is depressed a Drug (3) (not shown) filled Cartridge (4) (not shown) is first punctured by the non-patient end of Injection Needle (6) (not shown) and then the patient end of Injection needle (6) is inserted into the injection site. Drug (3) (not shown) is then injected (FIG. 1C). Once the injection is complete, the Device (1) is removed from the injection site and the Needle Shield (2) extends and locks out after extending past the Injection needle (6) tip, and prevent further use (FIG. 1D). In case of a multi-use autoinjector embodiment, the entire Needle Shield (2) containing the injection needle (6) can then be removed from the Device (1) and disposed of (FIG. 1E).

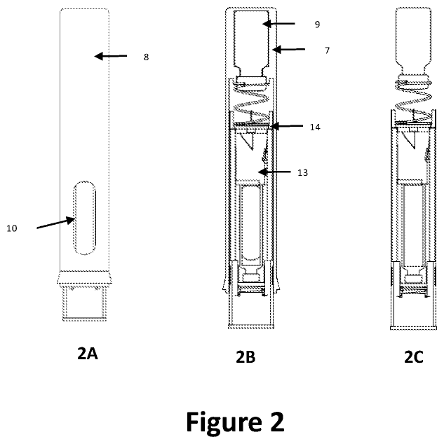

An autoinjector is depicted in FIG. 2, where the autoinjector device has a Housing (7) and Cover (8) that feature a Window (10) for Drug (3) inspection while containing the internal components. The Cover (8) is removed in FIG. 2B, and both the Cover (8) and Housing (7) are removed in FIG. 2C to show compressed gas source (9), Needle Disk (14) and Slider (13).

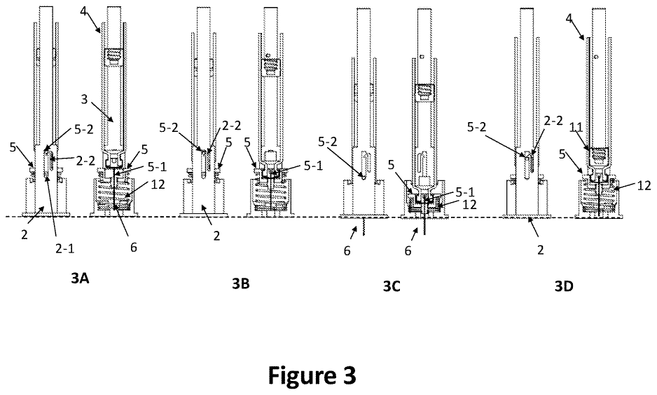

Isolated components of an autoinjector are depicted in FIG. 3 paired with corresponding sectional view, which depict how the Needle Shield (2) and Needle Holder (5) interact to pierce the Drug (3) filled Cartridge (4) and insert the Injection needle (6) as the Needle Shield (2) is depressed in one motion. Paired side and section views are shown across steps of use. The Needle Holder (5) contains a two-sided Needle (5-1), non-patient side to puncture the Cartridge Septum (4-1) and the patient side for injection. Also disposed within the Needle Holder is a Reservoir (5-3) for containing a sanitizing fluid (not shown), which is encase by a film (not show) adjacent to the Cartridge Septum (4-1). This film is removed upon removal of the cap exposing the sanitizing fluid to contact the Cartridge Septum (4-1). The Needle Holder (5) also features two radially directed Pins (5-2) that are contained in a Track (2-1) on the Needle Shield (2); the two parts are hence axially keyed. The injection procedure begins with the Device (1) in the as shipped (prior to use) position (FIG. 3A). As the Needle Shield (2) is depressed to start the injection (FIG. 3B), the Needle Shield Beam (2-2) initially blocks the Needle Holder Pin (5-2) from freely moving in the Track (2-1). The Beam (2-2) is configured such that the force for the Pin (5-2) to deflect the Beam (2-2) and move past it is higher than the force for the Needle (5-1) of the Needle Holder (5) to penetrate the Cartridge Septum (4-1). This results in no relative motion between the Needle Shield (2) and Needle Holder (5) until the Cartridge Septum (4-1) has been fully penetrated. Once the Cartridge Septum (4-1) has been fully penetrated, the Needle Holder (5) will be against the front of the Cartridge (4) forcing the Pin (5-2) to now deflect the Beam (2-2) and reach the bottom of the Track (2-1) (FIG. 3C). This relative motion of the Needle Holder (5) to the Needle Shield (2) inserts the Injection needle (6) into the injection site. Once injection is complete and as the Device (1) is removed (FIG. 3D), the Spring (12) decompresses and the Pin (5-2) moves back up the Track (2-1) enough for the Injection Needle (6) to be covered by the Needle Shield (2). The Needle Shield (2) and Needle Holder (5) then can be removed as one from the Device (1).

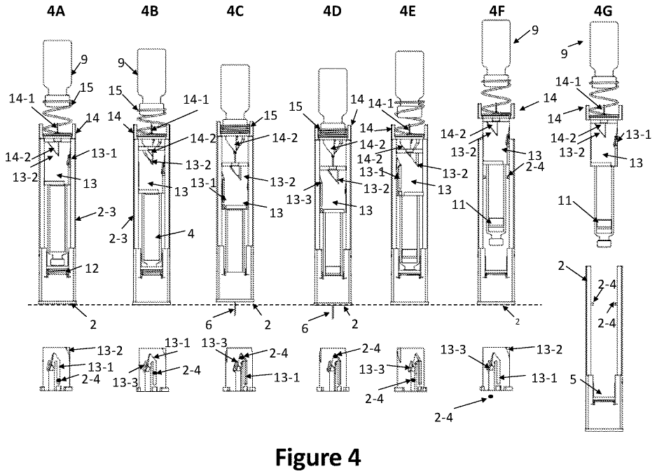

An autoinjector is depicted in FIG. 4, which illustrates how the Needle Shield (2), Slider (13), and Needle Disk (14) interact to lock-out the autoinjector Device (1) at the end of the dose delivery and allow for the Needle Shield (2) containing the Needle (6) to be removed. Front views of internal components are shown across steps of use. Below each front view is a paired side view of an isolated Slider (13) depicting the location of the Needle Shield Pin (2-4) in the Slider Track (13-1). The Needle Shield (2) interacts with the Needle Disk (14) via two Arms (2-3) that extend to push the Needle Disk (14) as the Needle Shield (2) is depressed. The two Needle Shield Arms (2-3) also both feature a Pin (2-4) that fits into the Slider Track (13-1) to control the rotational position of the Slider (13). The rotational position of the Slider (13) is additionally controlled by the Needle Disk Key (14-2) which is fitted to the Slider Keyway (13-2). The Slider (13) can only be rotated around the Cartridge (4) and is axially fixed. The Needle Shield (2) and Needle Disk (14) are both rotatably fixed but able to translate axially. The injection procedure begins with the Device (1) in the as shipped position (FIG. 4A). As the Needle Shield (2) is depressed to perform the injection (FIG. 4B), the Needle Shield Pin (2-4) travels upward in the Slider Track (13-1). The Needle Disk Key (14-2) also completely leaves the Slider Keyway (13-2) as it approaches the Canister (9). When the Needle Shield (2) has been fully depressed, the Pin (2-4) will reach the top of the Track (13-1) and force the Slider (13) to rotate due to its slanted geometry, as shown in the start of dose position (FIG. 4C). Once injection is completed (FIG. 4D), and as the Device (1) is removed from the injection site (FIG. 4E), the Needle Disk Spring (15) and Needle Shield Spring (12) decompress, and the Needle Shield (2) extends outward. Due to the Slider (13) having rotated, the Pin (2-4) now travels down a different path than it started from as the Needle Shield (2) extends outward. In this path, the Pin (2-4) is pushed past the Slider Beam (13-3) which deflects out of the way before returning and blocking the way back for the Pin (2-4). This blockage prevents the Needle Shield (2) from being depressed again and locks the Injection needle (6) from further use at the end of the dose delivery. Furthermore, the Track (13-1) is open-ended past the Beam (13-3) which allows for the Needle Shield (2) and contained Needle (6) to be entirely removed from the Device (1) by the user (FIG. 4F, FIG. 4G). The rotated position of the Slider (13) at the end of procedure position (FIG. 4E) also prevents the Needle Disk (14) from returning to its original position due to the Key (14-2) now being mismatched from the Keyway (13-2). The Key (14-2) instead pushes against the Keyway (13-2). The slanted edges of the Key (14-2) and Keyway (13-2) would cause the Slider (13) to rotate back to the as shipped position but the Pin (2-4) in the Track (13-1) prevents this. Only once the Needle Shield (2) has been fully removed can the Needle Disk (14) rotate the Slider (13) back to its as shipped position while also returning to its own as shipped position (FIG. 4F, FIG. 4G).

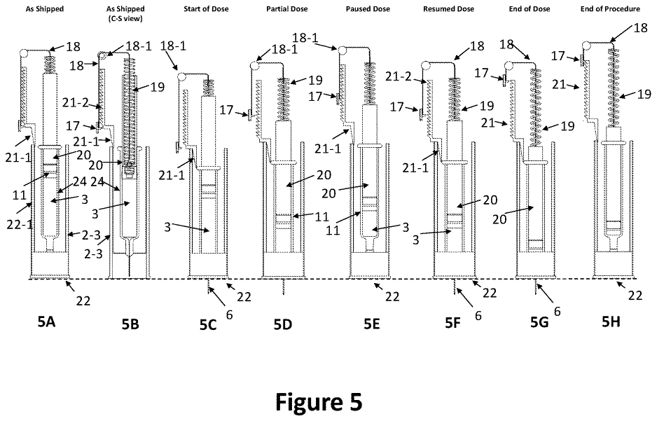

An autoinjector is depicted in FIG. 5, which shows a mechanism to pause a spring powered injection. The mechanism consists of an Indicator (17) with a Wire (18) that tethers it to the Spring (19) driven Plunger Rod (20). There is a Ratchet (21) that is actuated by the Needle Shield Arms (22-1) to either allow free travel of the Indicator (17) or to hold it in place. The Device (16) is received in the As Shipped position (FIG. 5A). Injection first begins when the Needle Shield (22) is fully actuated as seen in the Start of Dose position (FIG. 5C). This triggers the initial release of the Spring (19) to drive the Plunger Rod (20), while also removing the Ratchet (21) from the Indicator's (17) travel path due to the Needle Shield Arms (22-1) pushing on the Ratchet Ramp (21-1). To pause injection at any point, the device can be lifted from the injection site so the Needle Shield (22) is re-extended (FIG. 5E). When this occurs, the Needle Shield Arm (22-1) retracts from the Ramp (21-1) so that the Ratchet (21) moves to block the Indicator's (17) path, and the Indicator (17) slots in against one of the Ratchet's Teeth (21-2). With the Indicator (17) now unable to move, the Wire (18) is pulled taut and prevents further decompression of the Spring (19) so the Plunger Rod (20) stops dosing. The Device (1) remains in the paused position until the Needle Shield (22) is fully actuated again and the Ratchet (21) is cleared from the Indicator's (17) path, allowing injection to resume (FIG. 5F). The Device (16) can be paused multiple times during injection and at any point during injection. Only once the Device (16) has reached the End of Dose position (FIG. 5G) and the Device (16) is removed to end the procedure (FIG. 5H) does the Needle Shield (22) lock to prevent further use.

An alternative embodiment of the mechanism depicted in FIG. 5 can instead lock the rotation of the Pulley (18-1) via the Needle Shield Arms (22) instead of having the Ratchet (21) component. This would achieve an equivalent effect to blocking the Indicator (17) from travel. A wireless embodiment could also be made where the Ratchet (21) directly interfaces with a feature on Plunger Rod (20) to block its motion. In another alternative embodiment, the Pulley (18-1) is replaced by an alternate mechanism disposed coaxially with the hollow Plunger Rod (20).

Device (23) illustrates an embodiment with modular architecture designed to allow easy assembly of a syringe into an assembled autoinjector. The modular architecture consists of a top and bottom subassembly that can be separated by rotating them relative to each other, then pulling them apart. A syringe can then be placed inside the lower assembly and the top assembly can be pushed back on then turned to lock the subassemblies together. The assembly of the syringe into the autoinjector may now occur as the last step in the assembly process. This would allow for the syringe assembly to easily occur at a different manufacturing site than the rest of the Device (23) assembly.

Device (23) also illustrates an embodiment of an autoinjector with a dose dependent lockout. This allows for the autoinjector to utilize the ability to pause the injection while still locking out when injection is completed. The embodiment also allows for a larger viewing window of both the syringe and secondary dose progress indicator and locks out the device with a stronger lock compared to previously described embodiments. A biasing element between the compressed gas source and the penetrating component has also been removed without loss of function.

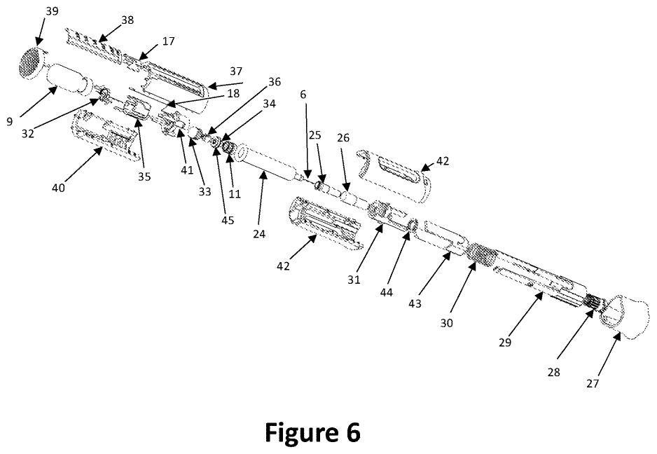

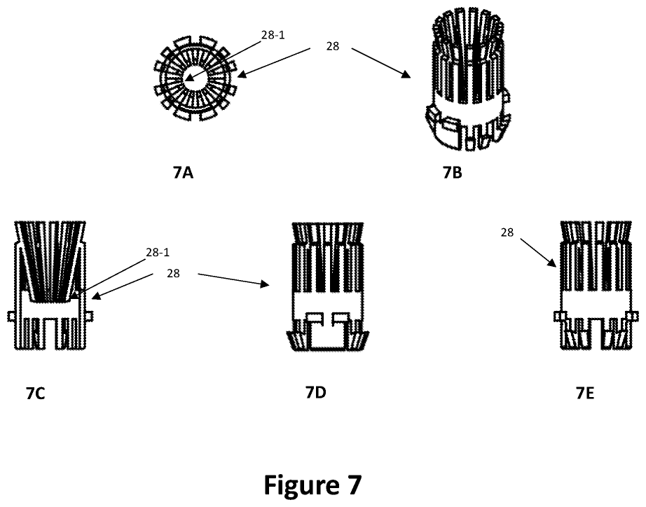

An autoinjector is depicted in FIG. 6, where the Device (23) houses a Syringe (24) that contains an injectable Drug (3) and has a Needle (6) to deliver the Drug (3). The needle (6) is enclosed by an elastomeric Needle Cover (25), which in turn is encased by a Rigid Needle Shield (26) that seals and covers the Needle (6) end of the Syringe (24). The Device (23) has an outer Cap (27) that is removed by the user just prior to injection. Removal of the Cap (27) also results in removal of the Needle Cover (25) and Rigid Needle Shield (26) via a Puller (28) to expose the injection Needle (6). The Puller (28) (FIG. 7) shown has Barbs (28-1) oriented to allow axial locking between of the Rigid Needle Shield (26) and the Puller (28) during assembly.

Removing the Cap (27) also reveals the Safety (29). The Safety (29) acts as the trigger to actuate injection. When the Safety (29) is axially depressed against the injection site, injection begins. When lifted from the injection site, the Safety (29) re-extends via a Spring (30) and injection stops. If lifted before all of the Drug (3) has been delivered, the Safety (29) returns to its initial position and can be re-depressed to resume the injection. If lifted after all of the Drug (3) has been delivered, the Safety (29) extends past its initial position and locks out, guarding the Needle (6). At lock out, the axial position of the Safety (29) is further away from the needle (6) tip than the same before the start of injection (initial position).

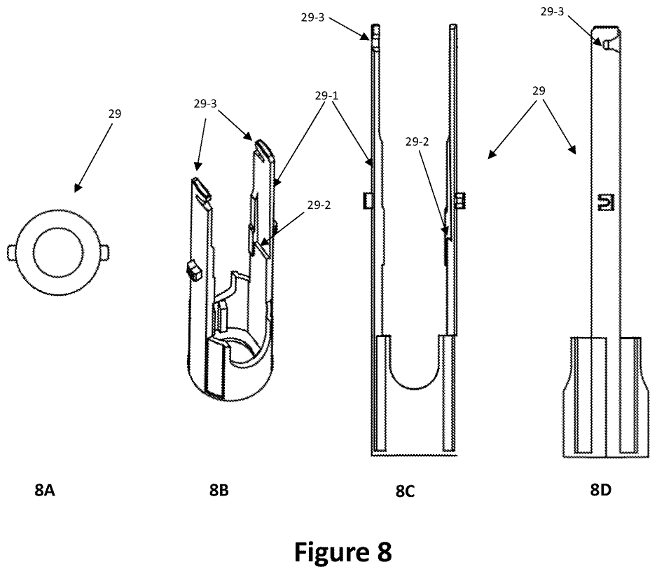

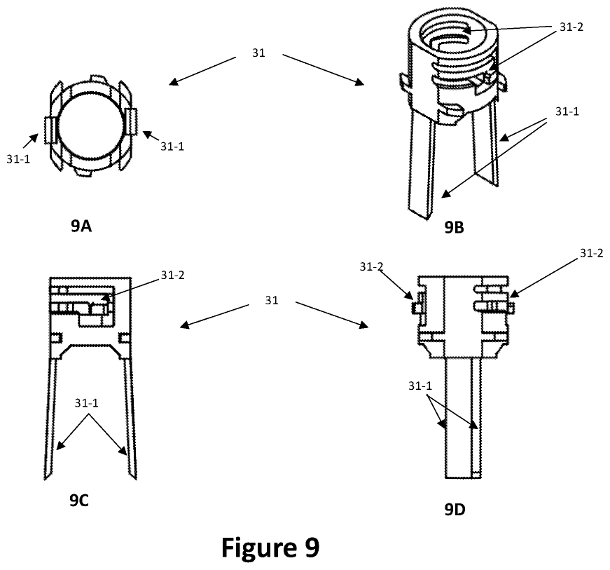

Each Arm (29-1) of the Safety (29) (FIG. 8) features a Stop (29-2) that interacts with a Beam (31-1) on the Locking Sleeve (31) (FIG. 9) to lock its position. Prior to lockout, the Beams (31-1) are tucked inside of the Stops (29-2) and allow free axial motion of the Safety (29). But when the Safety (29) extends past its initial position, a point of no return is reached, and the Beams (31-1) can flare radially outward and contact the Stops (29-2) to disable the Safety (29) from being axially depressed again.

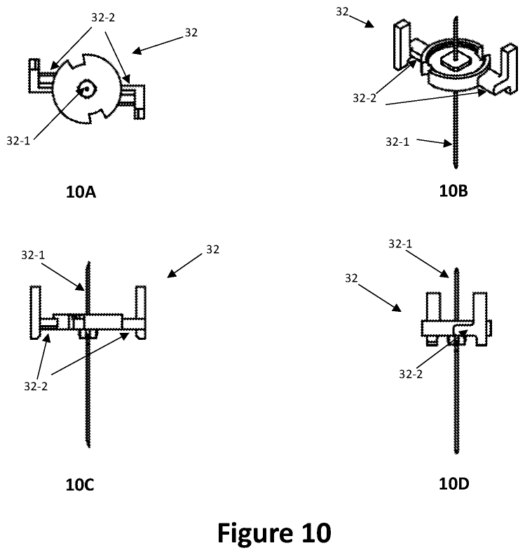

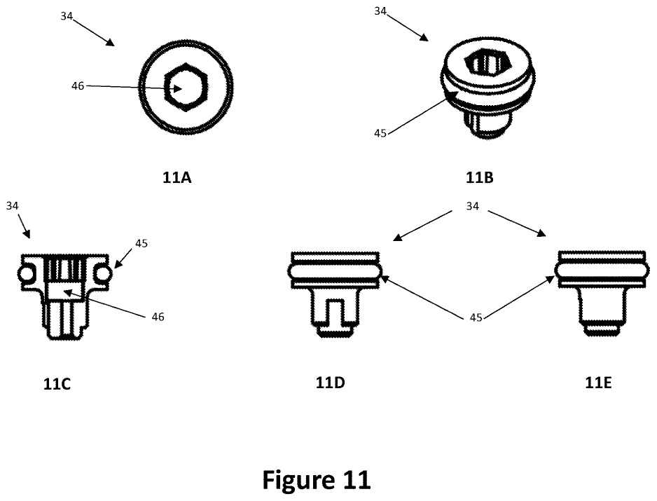

The Safety (29) controls injection by actuating the Needle Disk (32) (FIG. 10) axially. The Safety (29) features Hooks (29-3) at the end of each Arm (29-1) that fit onto Flanges (32-2) of the Needle Disk (32) during assembly. Axial motion of the Safety (29) directly translates to axial motion of the Needle Disk (32). When depressed, the Safety (29) slides the Needle Disk (32) towards the Canister (9) containing compressed gas. The Air Needle (32-1) of the Needle Disk (32) penetrates the Septum (9-1) of the Canister (9) and compressed gas flows through the Air Needle (32-1) into the sealed region between Syringe Seal (33) and the Follower (34) (FIG. 11). The pressure pushes the Follower (34) against the Plunger Stopper (11) and delivers the Drug (3). When the Safety (29) is re-extended, the Needle Disk (32) slides away from the Canister (9) and the Air Needle (32-1) is removed from the Septum (9-1) which reseals. The now exposed end of the Air Needle (32-1) simultaneously vents the pressure pushing the Follower (34) and injection stops. If Drug (3) remains in the Syringe (24), the still unlocked Safety (29) can be re-depressed to re-actuate release of the compressed gas and resume injection. If all the Drug (3) has been delivered, the Safety (29) locks and the Needle Disk (32) can no longer be actuated.

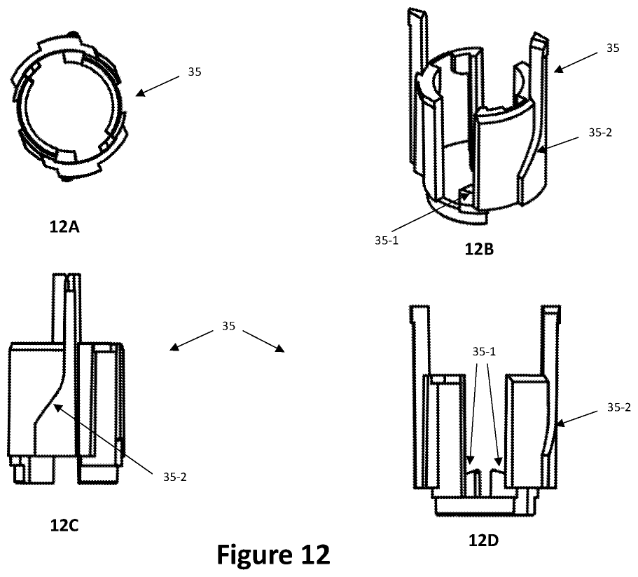

The extended position of the Safety (29) is controlled by interaction between the Needle Disk (32) and the Cam (35) (FIG. 12). The Flanges (32-2) of the Needle Disk (32) initially sit on two Stops (35-1) of the Cam (35). The Stops (35-1) prevent the Needle Disk (32) and Safety (29) from extending further out. The Needle Disk (32) is able to only travel between the Stops (35-1) and the Canister (9) until all of the Drug (3) has been delivered and the Cam (35) rotates to the lockout position. In the lockout position the Stops (35-1) of the Cam (35) are rotated out of the path of the Needle Disk (32) and it can extend out past its starting position, allowing the Safety (29) to extend out to its locked position. This interaction between the Cam (35) and Needle Disk (32) controlling the extended position of Safety acts as the pin and track from previously described embodiments. Where the Flanges (32-3) act as the pin, the Cam (35) and Stops (35-1) act as the track but the point of no return is now achieved with the Safety Stops (29-2) and the Locking beams (31-1).

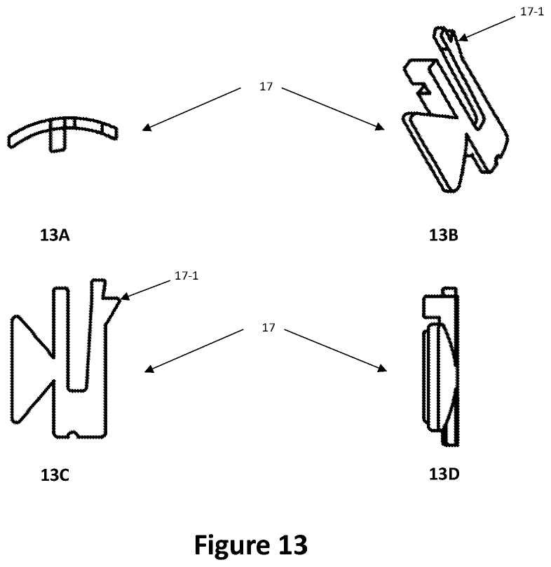

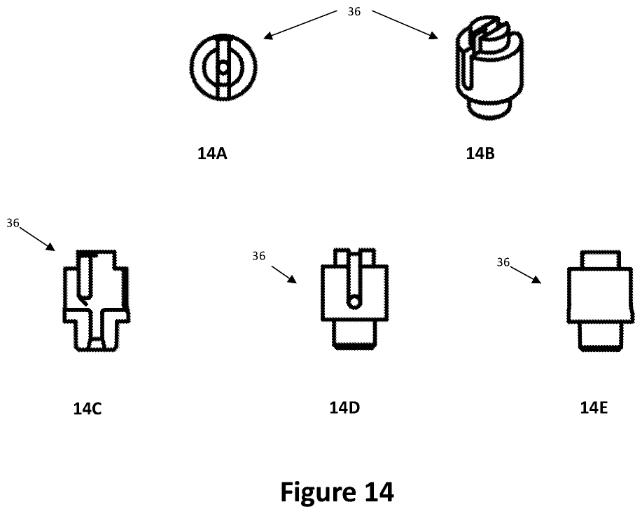

The rotation of the Cam (35) is triggered by the Indicator (17) (FIG. 13) that is tethered to the Plug (36) (FIG. 14) fitted in the Follower (34). The tether in this embodiment is via a thin Wire (18) that can pass through the Syringe Seal (33). As the Follower (34) advances in the Syringe (24), the Indicator (17) also advances. Near the end of injection, the Indicator (17) reaches a Ramp (35-2) on the Cam (35) and pushes against it, driving the rotation.

The Indicator (17) axially travels in a Track (37-1) on the Back Cover (37) during injection. At all times the Indicator Cover (38) physically shields the Indicator (17) from the user. However, the Indicator (17) can be seen translating underneath the partially clear Indicator Cover (17) and visually aligning with its Markings (38-1) to provide visual feedback of injection progress. The Indicator (17) also has a Flexible Beam (17-1) that is actuated as it travels by features on the Back Cover (37) or Indicator Cover (38) to provide an audible click heard by the user that signals the start and the end of injection.

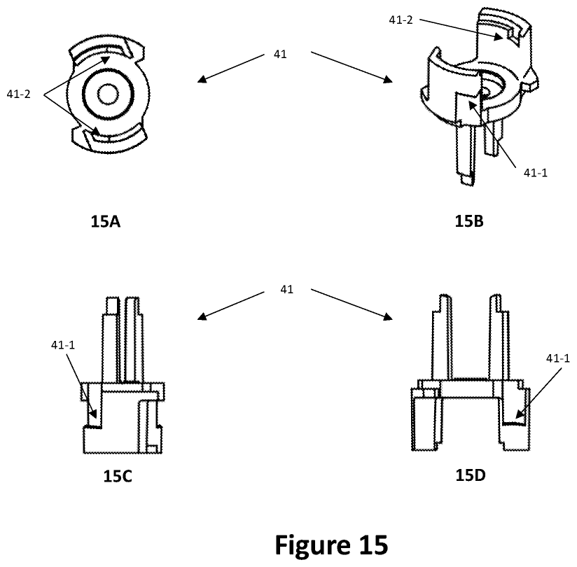

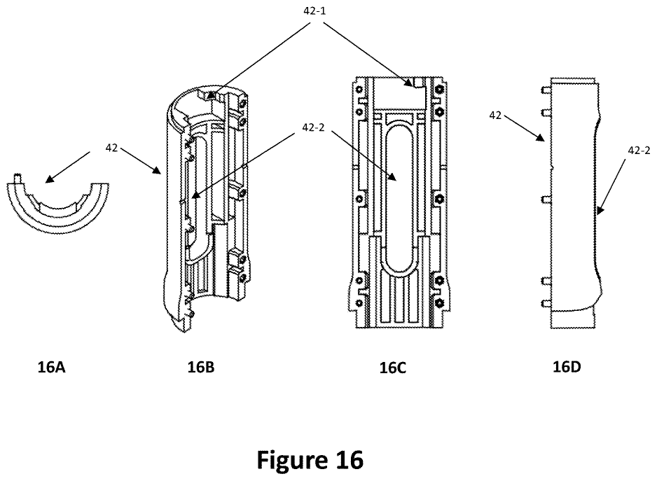

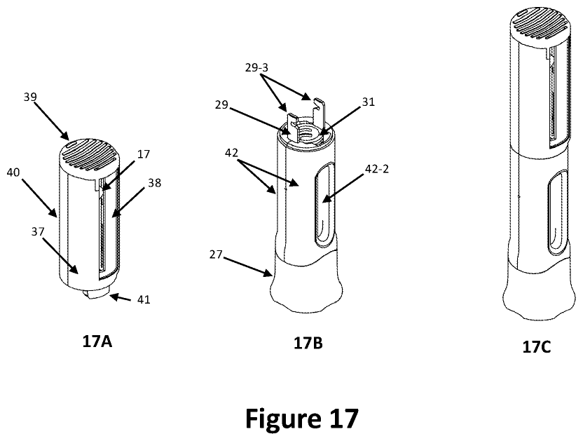

The Back Cover (37) assembles with the End Cap (39) to enclose the Indicator (17) and Indicator Cover (38). The Back Housing (40) also assembles with the Back Cover (37) and End Cap (39) to enclose the Canister (9), Cam (35), Needle Disk (32), and Brace (41). Alternate embodiments could feature the Back Housing (40), Back Cover (37), and Brace (41) as one component. The Brace (41) (FIG. 15) features a Lip (41-1) that hooks onto a Lip (42-1) in Front Housing (42) (FIG. 16) to join the front and back halves of the Device (23) (FIG. 17).

Within the Front Housing (42) the Syringe (24) is supported at its Shoulder (24-1) by the clear Window Sleeve (43) during injection. A compliant X-Ring (44) is also placed between the Window Sleeve (43) and Syringe (24) as a cushion. Both the Window Sleeve (43) and Locking Sleeve (31) are rotatably and axially fixed in the Front Housing (42), shown here in two halves. Alternate embodiments could have the Front Housings (42) as one whole component. Alternate embodiments could also have the Locking Sleeve (31) and Window Sleeve (43) as one component.

The Front Housings (42) also have a Window opening (42-2) that allows the user to view the Syringe (24) and Plunger Stopper (11) through the clear Window Sleeve (43).

The Brace (41) features an L-slot (41-2) that interacts with the L-Beam (31-2) of the Locking Sleeve (31) to rotatably fix the front and back halves of the Device (23) together only in the presence of a Syringe (24).

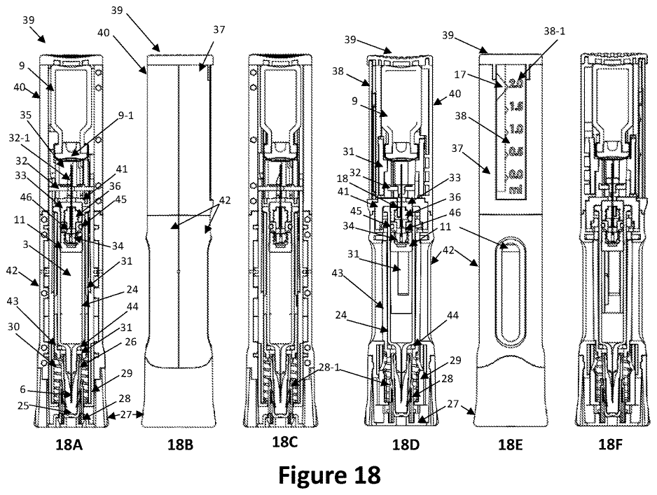

An autoinjector is depicted in FIG. 18 which shows section views of the complete Device (23) as assembled in the state that the user would receive it. FIG. 18A shows the left section view, and FIG. 18B and FIG. 18C show the right section. FIG. 18D shows the left section view, and FIG. 18E and FIG. 18F show the right section.

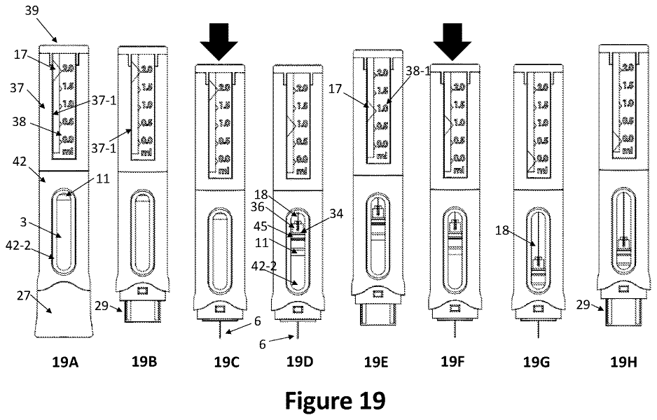

An autoinjector is depicted in FIG. 19, which shows the Injection Pause™ feature and stages of use in an external front view. The embodiment is shown from the front in FIG. 19A in the state that the user would receive the device. The Plunger Stopper (11) is visible through the Window (42-2). The Indicator (17) can also be seen at the start of its Track (37-1) next to a Marking (38-1) indicating the volume of Drug (3) in the Device (23). In FIG. 19B, the Cap (27) has been removed and the Device (23) is ready to use. FIG. 19C shows the Device (23) with the Safety (29) depressed to begin the injection. FIG. 19D shows the Device (23) midway through the injection. The Plunger Stopper (11) and Indicator (17) have both visibly advanced. The Follower (34), O-Ring (45), Plug (36), and Wire (18) are also now visible in the Window (42-2). In FIG. 19E the Device (23) has been lifted to pause the injection. The Safety (29) has re-extended to its initial position, but the Indicator (17) and Plunger Stopper (11) are paused at their respective midpoints. Then in FIG. 19F the Safety (29) is again depressed to resume the injection until the end of dose state is reached in FIG. 19G. The Indicator (17) and Plunger Stopper (11) have now reached the end of their travel. The Device (23) is then lifted off the injection site in FIG. 19H and the Safety (29) extends outward further than its initial position. The Safety (29) locks to shield the injection Needle (6) and the procedure is finished.

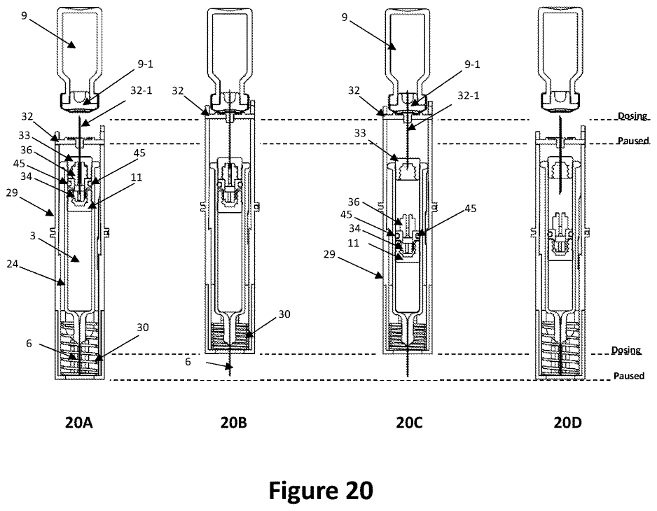

An autoinjector is depicted in FIG. 20, which includes only components directly relevant to the gas flow and sealing of the Injection Pause™ feature. Select stages of use are shown with an internal front view. FIG. 20A shows the ready to use state of the autoinjector. The double-sided Needle (32-1) of the Needle Disk (32) can be seen. The top tip of the Needle (32-1) is at a distance from the Canister (9) Septum (9-1). The bottom tip of the Needle (32-1) sits past a Seal Insert (46) assembled between the Follower (34) and Plug (36). The bottom tip of the Needle (32-1) also passes through the Syringe Seal (33). The Needle (32-1) never leaves the Syringe Seal (33). In this state the Needle (32-1) allows airflow from directly behind the Plunger Stopper (11) to the atmosphere. This allows the sealing Follower (34) to be assembled into the Syringe (24) without trapping air.

When the Safety (29) is depressed in FIG. 20B, the Needle Disk (32) is advanced until it is against the Canister (9) and the top tip of the Needle (32-1) penetrates the Septum (9-1). Immediately prior, in the same motion, the bottom tip of the Needle (32-1) is removed from the Seal Insert (46) which then reseals. This places the bottom tip of the Needle (32-1) in a sealed region of the Syringe (24) bounded by the Follower (34), O-Ring (45), and Seal Insert (46) at one end and the fixed Syringe Seal (33) at the other. Compressed gas flows from Canister (9), through the Needle (32-1), and into the sealed region of the Syringe (24). The pressure advances the Follower (34) as shown in FIG. 20C and Drug (3) is delivered. In FIG. 20D the autoinjector has been lifted in the middle of the injection and the Safety (29) has re-extended. This re-extension returns the Needle Disk (32) back to its initial position. The top tip of the Needle (32-1) is removed from the Canister (9) which reseals, and the tip is again exposed to the atmosphere. However, as the Follower (34) containing the Seal Insert (46) has axially advanced, the bottom tip of the Needle (32-1) remains in the pressurized region of the Syringe (24). This results in the pressure venting out through the Needle (32-1) and pausing of the Injection. The user can then resume injection by depressing the Safety (29) again to repressurize the Syringe (24).

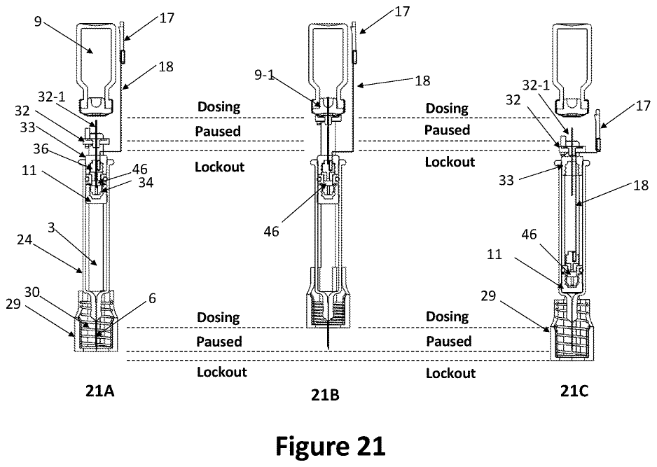

An autoinjector is depicted in FIG. 21, which highlights the wire path of the Injection Pause™ feature and select stages of use in an internal side view including only directly relevant components. The Wire (18) connects to the Indicator (17), passes through the Syringe Seal (33), and connects to the Plug (36) fitted in the Follower (34). FIG. 21A shows the autoinjector prior to injection, FIG. 21B shows the start of injection, and FIG. 21C shows the removed and locked device after a completed injection. The progression of the Wire (18) pulling the Indicator (17) over the course of the injection is shown. Those skilled in the art would appreciate that wire could be substituted by smooth cylindrical rod with appropriate modifications utilizing the same concept disclosed herein.

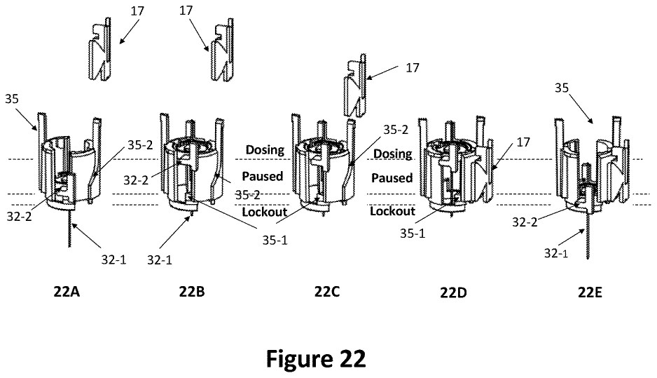

Isolated components of an autoinjector are depicted in FIG. 22. The Cam (35) actuation of the Injection Pause™ feature is shown with the Indicator (17) and Needle Disk (32) in isolation from the rest of the device. In FIG. 22A, the components are in their as shipped positions. The Needle Disk (32) is biased against the Stops (35-1) of the Cam (35) from the Safety (29) (not shown) and Spring (30) (not shown) extending it. In FIG. 22B The Safety (29) has been depressed and has pushed the Needle Disk (32) into the Canister (9) to begin injection. The Indicator (17) begins to advance as shown in FIG. 22C. In FIG. 22D the Indicator (17) has reached the end of its axial travel and has rotated the Cam (35) by pushing against the Ramp (35-2). This moves the Stop (35-1) of the Cam (35) out of the Needle Disk's (32) return path while the Needle Disk (32) is still in the Canister (9). In FIG. 22E the Device (23) has been removed from the injection site and the Safety (29) has re-extended. With the Cam (35) rotated and the Stop (35-1) moved, the Needle Disk (32) returns to a new position that is lower than its initial position. This results in extension of the Safety (29) past the point of no return and to trigger lockout.

The point in the injection at which the Indicator (17) rotates the Cam (35) could be configured to be any point during injection. A defeatured embodiment without the ability is also possible by configuring the Needle Disk (32) or Safety (29) to directly rotate the Cam (35) upon actuation of the Device (23).

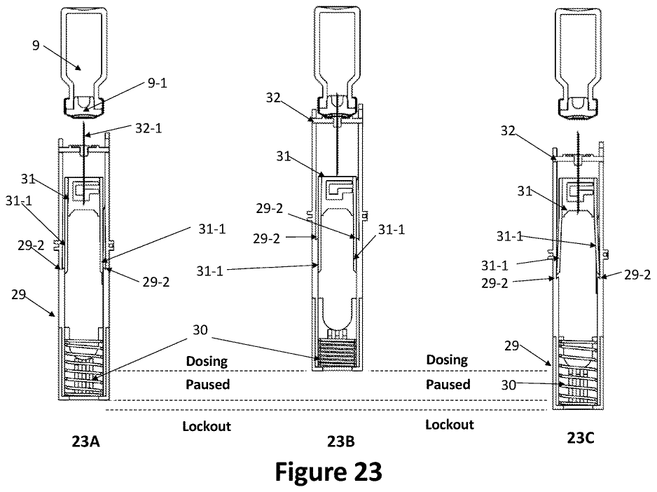

An autoinjector is depicted in FIG. 23, where isolated components of the lockout of the Injection Pause™ feature are shown in an internal front view. FIG. 23A shows the state of the device prior to injection or while injection is paused, when the Safety (29) is free to be depressed. In this state the Lockout Beams (31-1) are flexed radially inward and extend past the Stop (29-2) on each Arm (29-1) of the Safety (29). FIG. 23B shows the Device (23) during injection. The Safety (29) is depressed and the Lockout Beams (31-1) are still extended past the Stops (29-2) of the Safety (29). Releasing the Safety (29) prior to the end of injection returns the Device to the state in FIG. 23A. Releasing the Safety (29) only at the end of injection results in the locked-out state shown in FIG. 23C. In FIG. 23C the Safety (29) has extended past its initial position allowing the Stops (29-2) to pass the Lockout Beams (31-1) and flare radially outward. If the Safety (29) were pressed in this state each Stop (29-2) would collide with a Beam (31-1). Additionally, the Stops (29-2) and Locking Beams (31-1) are both angled to prevent the slippage when pressed. The device is completely locked out as a result.

An autoinjector is depicted in FIG. 17, which shows the Syringe Assembly and Subassemblies in an isometric external view. This embodiment of the device consists of an upper/back (FIG. 17A) and lower/front subassembly (FIG. 17B) that are then joined together (FIG. 17C). The Brace (41) extends from the upper subassembly to interface with the lower subassembly and the Safety (29) extends from the lower subassembly to interface with the upper subassembly.

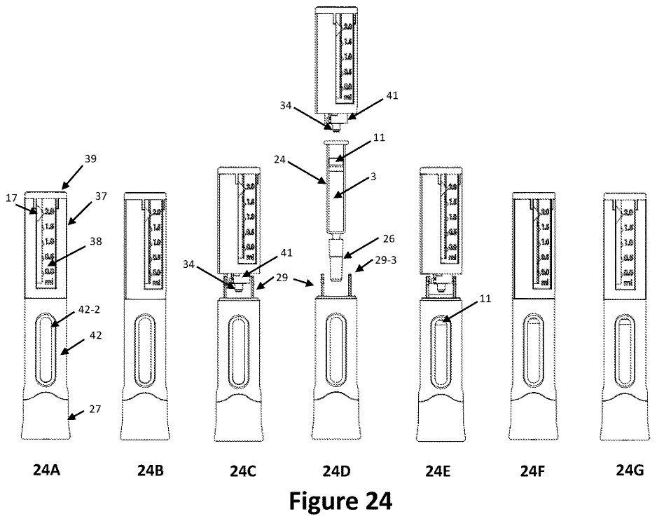

An autoinjector is depicted in FIG. 24, which shows a front external view of the steps to assemble a Syringe (24) into the Device (23). FIG. 24A shows the state of the Device (23) when received with no Syringe (24). The absence of a Syringe (24) can be verified through the Window (42-2). When no Syringe (24) is present the Device (23) can be separated into its subassemblies by first rotating the top subassembly as shown in FIG. 23B then lifting it up as shown in FIG. 23C. The Syringe (24) can then be axially slid into the bottom subassembly as shown in FIG. 24D and FIG. 24E. The subassemblies are then pushed back together (FIG. 24F) and rotated to their initial position (FIG. 24G). With a Syringe (24) now enclosed, the subassemblies rotatably lock together and will no longer separate. The presence of a Syringe (24) can be seen in the window.

The axial connection between the two subassemblies is provided by the Brace (41) Lip (41-1) interfacing with the Lip (42-1) in the Front Housings (42). The rotational lock between the subassemblies is independently provided from the L-shaped beam (31-2) on the Locking Sleeve (31) interfacing with the Syringe (24) and L-slot (41-2) of the Brace (41).

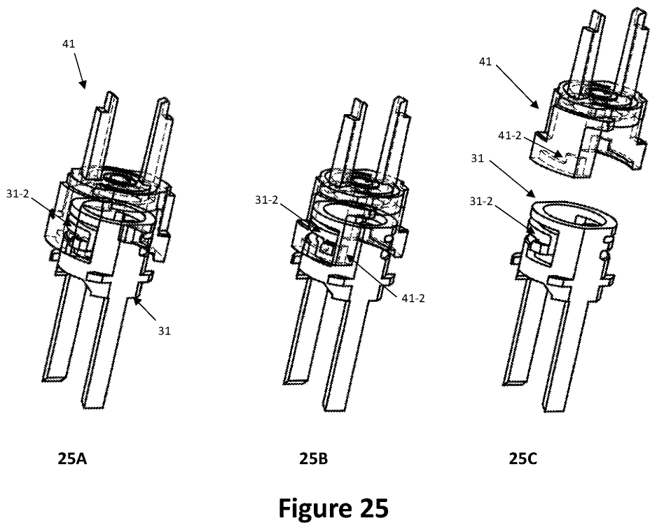

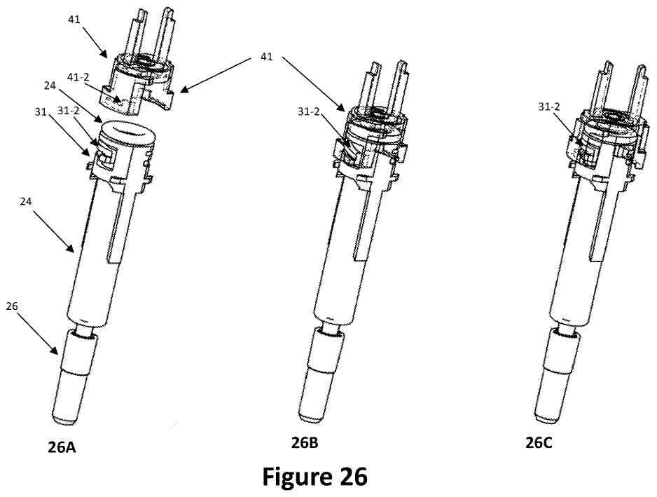

Isolated components of an autoinjector are depicted in FIGS. 25 and 26, which comprise the rotational lock for Syringe (24) assembly. FIG. 25 shows an isolated view of the Brace (41) and Locking Sleeve (31). The Brace (41) has hidden lines visible. In FIG. 25A the joined state of the Device (23) prior to Syringe (24) insertion is shown. The L-Beam (31-2) of the Locking Sleeve (31) is unflexed and the sits in the L-Slot (41-2) of the Brace (41). When the Brace (41) located in the top subassembly is rotated counterclockwise viewed from above in FIG. 25B, the L-Beam (31-2) of the Locking Sleeve (31) flexes radially inward and out of the L-Slot (41-2). This disengages the rotational lock between the subassemblies. Once rotated, the subassemblies can be axially separated (FIG. 25C.).

FIG. 26 shows an isolated view of the Brace (41), Locking Sleeve (31) and Syringe (24). In FIG. 26A the separated state of the Device (23) is shown with a Syringe (24) inserted. In FIG. 26B the two subassemblies are axially pushed together and the L-shaped Beam (31-2) is flexed downward by the Brace (41). In FIG. 26C the top subassembly containing the Brace (41) is rotated clockwise viewed from above, and the L-Beam (31-2) relaxes into the L-Slot (41-2). This completes the assembly of the Syringe (24) with the Device (23). With a Syringe (24) now present, the L-Beam (31-2) can no longer flex radially inward to disengage from the L-Slot (41-2) as in FIG. 25B, hence the subassemblies are rotatably locked.

The joining of the subassemblies flexes the L-Beam (31-2) downward, while the separating flexes the L-shaped Beam (31-2) radially inwards. This distinction allows for joining to occur unaffected by presence of a Syringe (24) while separating can only occur in absence of a Syringe (24). To this end, the L-Beam (31-2) is L-shaped so that forced rotation with a Syringe (24) present causes the L-Beam (31-2) to flex upward and jam rotation, rather than slip downward back along the joining path and unlock. This maintains the distinction between joining and separating.

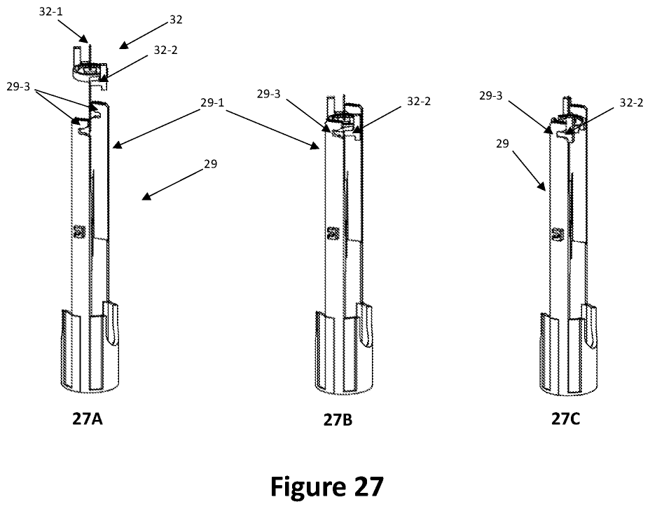

Isolated components of an autoinjector are depicted in FIG. 27. The Safety (29) and Needle Disk (32) are shown in an isolated view to highlight how the Safety (29) couples with the Needle Disk (32) when the top and bottom subassemblies are joined. FIG. 27A shows the Safety (29) and Needle Disk (32) while the subassemblies are axially separated. FIG. 27B shows the Safety (29) and Needle Disk (32) while the subassemblies are axially pushed together. The Hooks (29-3) on each Arm (29-1) of the Safety (29) slide past the Flanges (32-2) of the Needle Disk (32). In FIG. 27C The top subassembly containing the Needle Disk (32) is then rotated and the Flanges (32-2) of the Needle Disk (32) slot into the Hooks (29-3) of the Safety (29). The Safety (29) will now axially push and pull the Needle Disk (32) when actuated. This attachment is undone when the subassemblies are rotated and separated.

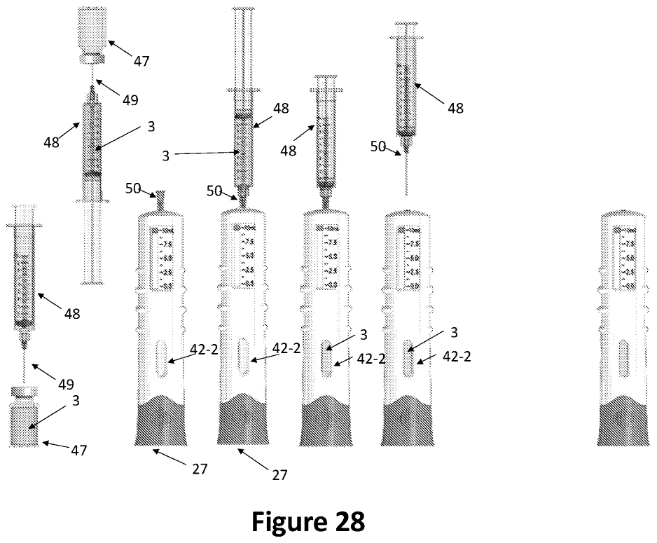

An autoinjector is depicted in FIG. 28, which shows a Drug (3) being loaded. The Drug (3) is provided in a Vial (47). This Drug (3) is then transferred into a Syringe (48). Alternatively, the drug may be provided in a prefilled syringe. The Drug (3) from the Syringe (48) is then transferred into the autoinjector through a removable, hollow Connector (50). The Connector (50) mates with a syringe and is typically a female luer or another configuration to achieve the same means. The autoinjector is now filled upon transfer of the drug from the Syringe (48) into the autoinjector through Connector 50. Once all of the Drug (3) is transferred into the autoinjector the Connector (50) is removed while still attached to the Syringe (48). This autoinjector along with the removable Connector (50) incorporated within it is provided sterile prior to filling. The Drug (3) filling could be conducted in a laminar flow hood depending on the duration of storage of drug after transfer into the autoinjector.

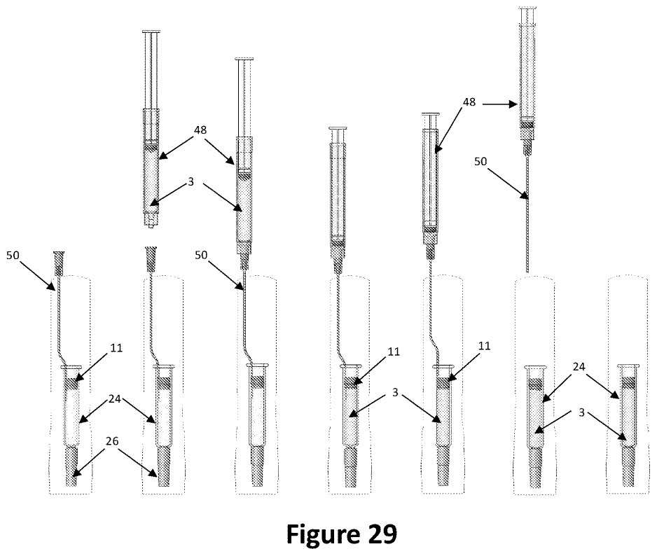

An autoinjector is depicted in FIG. 29, which shows a drug being loaded in an alternate embodiment. This figure shows how the Connector (50) is configured and bypasses the Plunger Stopper (11).

An autoinjector disclosed here includes a compressed gas source incorporated into multiple embodiments of an autoinjector. Embodiments disclosed herein aim to improve upon shortcomings of other compressed gas autoinjector technologies and other autoinjectors. Embodiments of the autoinjector include features to further improve usability and address some of the technology gaps in current autoinjectors. Novel features disclosed herein could be applied to autoinjectors not having a compressed gas power source.

This disclosure describes a compact, high performance autoinjector powered by a compressed gas source. This disclosure also provides methods of manufacturing an autoinjector with a compressed gas source consisting of a container having a closure element that pierceable, yet hermetically seals around the piercing element (such as a sharp, hollow metal tube/needle). When this tube is removed, the pierceable closure element seals again retaining the uncompressed, pressurized gas within the compressed gas source.

According to an aspect of this disclosure, an autoinjector is provided for use in the injection of an injectable drug with assistance of a compressed gas. The autoinjector includes a compressed gas source and a syringe mounted together by a housing. The compressed gas source includes a rigid container defining an interior space and an opening into the interior space, and a non-rigid sealing structure disposed and configured to seal the opening into the interior space to maintain the compressed gas under compression. The syringe includes a barrel, a syringe needle fluidly coupled to an interior of the barrel, and a plunger stopper disposed to translate within the barrel, a seal disposed to seal the barrel opposite the syringe needle. The plunger stopper is radially disposed within the barrel and separates the interior of the barrel into a drug space configured to contain the injectable drug between the plunger stopper and the syringe needle, and an actuation space between the plunger stopper and the seal. The autoinjector further includes a puncturing needle. The puncturing needle is axially aligned to selectively penetrate the non-rigid sealing structure of the compressed gas source upon relative axial movement between the puncturing needle and the compressed gas source to fluidly couple the puncturing needle with the compressed gas source. At least one of the compressed gas source and the puncturing needle is movably mounted whereby the puncturing needle selectively penetrates the non-rigid sealing structure to selectively fluidly couple the compressed gas source with the actuation space.

According to another aspect of this disclosure, there is provided a compact sealed compressed gas source. The compressed gas source includes a rigid container, a non-rigid sealing structure, a crimping sleeve, and a conically shaped rigid structure. The rigid container defines an interior space, and includes an enlarged neck portion defining an opening into the interior space. The non-rigid sealing structure is disposed and configured to seal the opening into the interior space. The non-rigid sealing structure is at least partially disposed within the opening into the opening. The crimping sleeve includes a generally cylindrical portion disposed around and crimped below the enlarged neck portion of the rigid container and a generally radially extending portion defining an aperture in alignment with the opening into the interior space. The crimping sleeve is disposed to resist outward movement of the non-rigid sealing structure from the enlarged neck. The conically shaped rigid structure is disposed to exert a sealing force against the non-rigid sealing structure. The conically shaped rigid structure may be formed by the crimping sleeve itself or by a separate structure, such as a conical washer. A compressed gas is disposed within the interior space of the rigid container.

According to further aspect of this disclosure, there is provided a method of manufacturing such a sealed compact gas source by inserting the non-rigid sealing structure into the opening into the interior space of the rigid container, disposing the crimping sleeve around the enlarged neck portion of the rigid container with the conically shaped rigid structure disposed to exert an inwardly directed sealing force on the non-rigid sealing structure, crimping the crimping sleeve around the enlarged neck portion, and charging the rigid container with a compressed gas.

According to yet another aspect of this disclosure, there is provided a method of administering an injectable drug by fluidly coupling an actuation space of a syringe with a compressed gas source to provide compressed gas to axially translate a plunger stopper within a barrel of the syringe to inject the injectable drug.

The components of an autoinjector provided for use in the injection of an injectable drug with assistance of a compressed gas as disclosed here may be modified and adapted to provide a spring-powered autoinjector.

Those of skill in the art will appreciate that other arrangements for locking out needle safety shield at the end of injection may be accomplished based upon the teachings of this disclosure. For example, a cam-based mechanism can be implemented incorporating concepts disclosed herein.

The autoinjector housings can be re-configured to split transversely to the axis of the device instead of the longitudinal split housing design to enable better manufacturability and assembly with a prefilled syringe.

It is envisioned that the slider in either embodiment could be placed coaxially with the syringe without an axial overlap, and yet result in the same outcome as articulated in the foregoing.

Incorporation of electronic communication components and use of the disclosed invention with electronic methods of data capture, management and transmission are envisioned as part of this disclosure. The plurality of systems including a drug delivery device, and the plurality of methods for using the drug delivery system, may involve the operation of the device in one or more stages or states.

These states may be determined through the use of one or more sensors in combination with one or more controllers. The sensors may rely on mechanical, electrical or chemical sensing mechanisms, and the controllers may be mechanical, electrical or electro-mechanical. By way of example and not by way of limitation, the states may relate to the operation of the drug delivery device, or to the condition of the drug delivery device. The system and methods may use the state determination to control the operation of the drug delivery device, and/or may communicate the state determination to other devices, such as third-party servers that may collect, process and/or further disseminate the state determinations received from the system including the drug delivery device, the one or more sensors, and the one or more controllers. In addition, or in the alternative, the systems and methods may communicate the state determination to local devices, such as a mobile computing device (e.g., cell phone).

The system or method according to the disclosure will determine one or more states relative to the drug delivery device. For example, the system or method may determine if the drug delivery device is in one or more operational states (i.e., a state relating to the operation of the drug delivery device to deliver the drug to the patient). A non-exhaustive list of the general operational states may include (i) packaged/ready for distribution; (ii) packaged/distributed; (iii) unpackaged/ready for administration; (iv) sterile barrier removed; (v) device applied; (vi) cannula injected (or inserted); (vii) drug delivery initiated; (viii) drug delivery completed; and (ix) device removed. The system or method may determine specific operational states within each of the general operational states; for example, the system or method may determine if plunger has been moved from a first end of a bore (defining a drug reservoir) to a second end of the bore to determine if the drug delivery device is in the “drug delivery complete” state.

Furthermore, the system or method may determine if the drug delivery device is in one or more condition states (i.e., a state relating to the condition of the drug delivery device, not necessarily related to the operation of the drug delivery device to deliver the drug to the patient). A non-exhaustive list of condition states may include (i) age (e.g., taken with respect to a manufacturing date or an expiration date); (ii) sterility/contamination; (iii) temperature (or temperature history); and (iv) orientation. The determination of a condition state may be considered as part of the determination of an operational state; for example, the determination of the temperature state may be considered as part of the determination of the “ready for administration” state. Alternatively, the operational and condition states may be determined separately.

One use for a needle insertion signal can be release of a delivery lockout once the needle has been inserted into the patient. Alternatively, if the completion of drug delivery occurs and the needle was inserted for the entire period of time between “delivery triggered” and “delivery completion” a very high degree of confidence in successful dosing is provided. And conversely if the timing of the events do not overlap appropriately it may be possible to predict the amount of dose that was successfully delivered based on the systems delivery characteristics. In the event that an incomplete or unsuccessful dose administration is detected and reported, there is significant incremental value if the amount of dose discrepancy is also reported. A “Smart Drug Delivery Device” might be used for many different types of medicaments with varying therapeutic effects and toxicity risk profiles. For example, some medications may require urgent completion of dosing such as by a second injection for any incomplete dose if there is a low risk of toxicity but high risk of complications with a missed or incomplete dose. Alternatively, a healthcare provider may prefer to know about a missed or incomplete dose but wait for the next dose instead of scheduling a replacement if the risk of complications is low. Importantly, there may be opportunities to mitigate issues associated with incomplete dosing by administering just the amount of missed dose if it is correctly recorded and reported, offering an opportunity to maximize benefit while minimizing the overall cost of care.

It will be appreciated by those skilled in the art that for the single dose embodiment, upon completion of dose delivery, the embodiment could be configured to empty entirety of the contents of the compressed gas source by emptying the non-drug chamber adjacent to the plunger stopper.

It will be appreciated that the foregoing description provides examples of the disclosed autoinjectors and techniques. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The above description describes various systems and methods for use with a drug delivery device. It should be clear that the system, drug delivery device or methods can further comprise use of a medicament listed below with the caveat that the following list should neither be considered to be all inclusive nor limiting. The medicament will be contained in a reservoir. In some instances, the reservoir is a primary container that is either filled or pre-filled for treatment with the medicament. The primary container can be a cartridge or a pre-filled syringe.

Examples of other pharmaceutical products for use with the device may include, but are not limited to, antibodies such as Vectibix® (panitumumab), Xgeva™ (denosumab) and Prolia™ (denosamab), Herceptin® (trastuzumab), RITUXAN HYCELA® (rituximab/hyaluronidase), Darzalex Faspro® (daratumumab and hyaluronidase), KEYTRUDA® (pembrolizumab), TALVEY® (talquetamab-tgvs), TECVAYLI® (teclistamab-cqyv) and the like; other biological agents such as Enbrel@ (etanercept, TNF receptor/Fc fusion protein, TNF blocker), Neulasta® (pegfilgrastim, pegylated filgastrim, pegylated G-CSF, pegylated hu-Met-G-CSF), Neupogen@ (filgrastim, G-CSF, hu-MetG CSF), and Nplate® (romiplostim); synthetic protein like COPAXONE® (glatiramer acetate) and the like; small molecule drugs such as Sensipar® (cinacalcet), Uzedy (risperidone) and the like. The device may also be used with a therapeutic antibody, a polypeptide, a protein, synthetic peptide or other chemicals, such as an iron, for example, ferumoxytol, iron dextrans, ferric glyconate, and iron sucrose. The pharmaceutical product may be in liquid form, or reconstituted from lyophilized form. As used here, an antibody, a polypeptide, a protein, and/or a synthetic peptide, include fusions, fragments, analogs, variants or derivatives thereof.

It should be noted that the configurations of the various embodiments of the drug delivery devices and drug delivery systems described herein are illustrative only. Although only a few embodiments of the of the drug delivery devices and drug delivery systems have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, orientations, etc.) without materially departing from the novel teachings and advantages of the subject matter of this disclosure. For example, any combination of one or more of the sensors and sensor systems described herein may be incorporated into one or more of the drug delivery systems and drug delivery devices described herein. Also, the order or sequence of any process or method steps described herein may be varied or re-sequenced, in any combination, according to alternative embodiments. Furthermore, any combination of one or more of the elements of one or more of the claims set forth at the end of this disclosure is possible.

Although the preceding text sets forth a detailed description of different embodiments of the invention, it should be understood that the legal scope of the invention is defined by the words of the claims set forth at the end of this patent. The detailed description is to be construed as exemplary only and does not describe every possible embodiment of the invention because describing every possible embodiment would be impractical, if not impossible. Numerous alternative embodiments could be implemented, using either current technology or technology developed after the filing date of this patent, that would still fall within the scope of the claims defining the invention.

It should also be understood that, unless a term is expressly defined in this patent using the sentence “As used herein, the term ‘x’ is hereby defined to mean ‘y’” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

[We/I] claim:

1. An autoinjector comprising:
a syringe including a barrel, a syringe needle fluidly coupled to an interior of the barrel, and a plunger stopper disposed to translate within the barrel, the plunger stopper radially disposed within the barrel, the plunger stopper separating the interior of the barrel into a drug space configured to contain the injectable drug between the plunger stopper and the syringe needle;
a slider co-axially disposed with the syringe, the slider being rotatably disposed relative to the syringe and substantially axially fixed relative to the syringe;
a member axially adjacent to the plunger stopper, the axial position of the member being synchronous with the axial position of the plunger stopper within the barrel;
a needle safety shield, the needle safety shield being slidably disposed relative to the syringe and the slider whereby an axial force exerted on the needle safety shield slides the needle safety shield relative to the syringe from a shielded position to actuate an applied force onto the plunger stopper wherein the syringe needle is not axially exposed to an injection position wherein the syringe needle is axially exposed to penetrate the injection site;
wherein the movement of the needle safety shield is at least partially guided by a pin and track arrangement, one of the pin and the track being formed with the needle safety shield and the other of the pin and the track being formed with the slider whereby movement of the pin within the track controls the position of the needle safety shield relative to the slider, the track including a point-of-no-return, the pin returning the needle safety shield to the shielded position when the axial force is removed when the pin is disposed in the track proximal to the point-of-no-return such that the needle safety shield remains axially movable to the injection position wherein the applied force is removed, and the pin moving to and locking the needle safety shield in the shielded position when the axial force is removed when the pin is disposed in the track distal to the point-of-no-return with rotation of the slider; and
wherein rotation of the slider is actuated by movement of the member moving synchronous with the plunger stopper, the member rotating the slider positioning the safety shield pin to be guided along the track including the point-of-no-return to axially lock the needle safety shield in the shielded position as the plunger stopper reaches the end of all dose delivery.

2. The autoinjector of claim 1 further comprising a status indicator indicative qualitatively and quantitatively of the progress of delivery of the injectable drug from the syringe.

3. The autoinjector of claim 2 wherein the status indicator comprises a dose indicator.

4. The autoinjector of claim 3 wherein the dose indicator is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe.

5. A method for dose fractioning by an autoinjector comprising the steps of:
retracting a needle safety shield from an unused state to actuate an autoinjector;
an applied force moving synchronously a member with a plunger stopper disposed to translate within a syringe or cartridge;
interrupting autoinjector operation by removing applied force on the plunger stopper translation of needle shield pin along original track of slider to its unused state removing the applied force;
resuming autoinjector operation by reapplying force on the plunger stopper, wherein the force is actuated by retraction of the needle safety shield;
completing autoinjector operation by removing applied force on the plunger stopper translation upon orienting a slider to guide a radial pin of the needle safety shield to a point of no return locking out the needle safety shield.

6. The method of claim 5 wherein the autoinjector further comprises a status indicator indicative qualitatively and quantitatively of the progress of delivery of the injectable drug from the syringe.

7. The method of claim 6 wherein the status indicator comprises a dose indicator.

8. The method of claim 7 wherein the dose indicator is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe.

9. An autoinjector comprising:
a cartridge including a barrel, a plunger stopper disposed to translate within the barrel, the plunger stopper radially disposed within the barrel, the plunger stopper separating the interior of the barrel into a drug space configured to contain the injectable drug between the plunger stopper and a cartridge crimped stopper;
a slider co-axially disposed with the cartridge, the slider being rotatably disposed relative to the cartridge and substantially axially fixed relative to the cartridge;
a member axially adjacent to the plunger stopper, the axial position of the member being synchronous with the axial position of the plunger stopper within the barrel;
a needle safety shield, the needle safety shield being slidably disposed relative to the cartridge and the slider whereby an axial force exerted on the needle safety shield slides the needle safety shield relative to the cartridge from a shielded position wherein the injection needle axially keyed to the needle safety shield is not axially exposed to an injection position wherein the injection needle is axially exposed to penetrate the injection site;
wherein the movement of the needle safety shield is at least partially guided by a pin and track arrangement, one of the pin and the track being formed with the needle safety shield and the other of the pin and the track being formed with the slider whereby movement of the pin within the track controls the position of the needle safety shield relative to the slider, the track including a point-of-no-return, the pin returning the needle safety shield to the shielded position when the axial force is removed when the pin is disposed in the track proximal to the point-of-no-return such that the needle safety shield remains axially movable to the injection position, and the pin moving to and locking the needle safety shield in the shielded position when the axial force is removed when the pin is disposed in the track distal to the point-of-no-return with rotation of the slider;
wherein rotation of the slider is actuated by movement of the member moving synchronous with the plunger stopper, the member rotating the slider positioning the safety shield pin to be guided along the track including the point-of-no-return to axially lock the needle safety shield in the shielded position as the plunger stopper reaches the end of all dose delivery;
wherein the force applied to the plunger stopper is counterbalanced by a component introduced in the path of the component coupled to the member when the pin travels from the injection position to the shielded position; and
wherein the needle safety shield with interlocked injection needle is removable after the safety shield pin is at the point of no return.

10. The autoinjector of claim 9, wherein a portion of the member is radially disposed within and sealed against the barrel.

11. The autoinjector of claim 9 further comprising a reservoir containing sanitizer that is disposed between the injection needle and the non-drug side of the cartridge crimped stopper and configured so that the injection needle passes through the reservoir prior to piercing the cartridge crimped stopper to be fluidly couple to the interior of the barrel when the needle safety shield moves from the shielded position to the injection position.

12. The autoinjector of claim 9 further comprising a status indicator indicative qualitatively and quantitatively of the progress of delivery of the injectable drug from the syringe.

13. The autoinjector of claim 12 wherein the status indicator comprises a dose indicator.

14. The autoinjector of claim 13 wherein the dose indicator is slidably disposed within a dose indicator window and the dose indicator is tethered to a member axially adjacent to and moving synchronous with plunger stopper such that the position of the dose indicator relative to the dose indicator window is indicative of an axial position of the plunger stopper relative to the syringe.

15. A sterile autoinjector comprising a preinstalled sterile container operably linked by a connector to a fluid port wherein the connector bypasses a plunger stopper radially disposed within and sealed against the container positioned to deliver a preset injection volume; wherein the connector and fluid port are configured to be removed once the container is filled to the preset injection volume; and wherein the plunger stopper is configured to be radially sealed once the connector is unlinked from the container.

16. The autoinjector of claim 15, wherein the connector is hollow tubing.

17. The autoinjector of claim 15, wherein the sterile container is a cartridge.

18. The autoinjector of claim 15, wherein the fluid port is a female luer.

19. The autoinjector of claim 15, wherein all other components involved in normal autoinjector operation are in a ready-to-use state prior to filling the container with an injectable fluid.
